(19) United States
(12) Patent Application Publication
(10) Pub. No.: US 2022/0175875 A1
(43) Pub. Date: Jun. 9, 2022

(54) METHODS OF TREATING CANCER BY TARGETING TUMOR-ASSOCIATED MACROPHAGES

(71) Applicants: ENDOCYTE, INC., West Lafayette, IN (US); PURDUE RESEARCH FOUNDATION, West Lafayette, IN (US)

(72) Inventors: Yingjuan J. LU, West Lafayette, IN (US); Leroy W. WHEELER, II, West Lafayette, IN (US); Philip Stewart LOW, West Lafayette, IN (US); Christopher Paul LEAMON, West Lafayette, IN (US)

(21) Appl. No.: 17/524,211

(22) Filed: Nov. 11, 2021

Related U.S. Application Data
(63) Continuation of application No. 15/529,392, filed on May 24, 2017, now abandoned.
(60) Provisional application No. 62/084,194, filed on Nov. 25, 2014.
(60) Provisional application No. 62/149,067, filed on Apr. 17, 2015.

(51) Int. Cl. A61K 38/08; A61K 49/00; A61K 47/55; A61K 47/54; G01N 33/574

(52) U.S. Cl. CPC A61K 38/08; A61K 49/0032; A61K 49/0056; G01N 2333/705; A61K 47/545; A61K 49/0052; G01N 33/57492; A61K 47/551

(57) ABSTRACT

Methods for treating cancers using one or more compounds comprising a folate receptor binding ligand attached to a drug via a linker are described. Methods for treating cancers using one or more compounds comprising a folate receptor binding ligand attached to a drug via a linker to target tumor associated macrophages are described.

### CROSS REFERENCE TO RELATED APPLICATIONS

This application claims priority under 35 U.S.C. § 119(e) to U.S. Provisional Application Ser. No. 62/084,194, filed Nov. 25, 2014 and U.S. Provisional Application Ser. No. 62/149,067, filed Apr. 17, 2015, in which all of which are incorporated herein by reference in their entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: One 4,096 byte ASCII (Text) file named “20150-244673_SL.txt” created on Nov. 24, 2015.

### FIELD OF THE INVENTION

The invention described herein relates to methods for treating cancers using one or more compounds comprising a folate receptor binding ligand attached to a drug via a linker. The invention described herein also relates to methods for treating cancers using one or more compounds comprising a folate receptor binding ligand attached to a drug via a linker to target tumor associated macrophages.

### BACKGROUND AND SUMMARY OF THE INVENTION

Despite the fact that there have been significant developments in anti-cancer technology, such as radiotherapy, chemotherapy and hormone therapy, cancer still remains the second leading cause of death following heart disease in the United States. Most often, cancer is treated with chemotherapy utilizing highly potent drugs, such as mitomycin, paclitaxel and camptothecin. In many cases, these chemotherapeutic agents show a dose responsive effect, and cell kill is proportional to drug dose. A highly aggressive style of dosing is thus necessary to eradicate neoplasms; however, high-dose chemotherapy is hindered by poor selectivity for cancer cells and severe toxicity to normal cells. This lack of tumor-specific treatment is one of the many hurdles that need to be overcome by current chemotherapy.

One solution to current chemotherapy limitations is to deliver a biologically effective concentration of an agent to tumor tissue with very high specificity. To reach this goal, much effort has been undertaken to develop tumor-selective drugs by conjugating anti-cancer drugs to hormones, antibodies, and vitamins. For example, the low molecular weight vitamin, folic acid, and other folate receptor binding compounds and ligand are especially useful as targeting agents for folate receptor-positive cancer cells and tumors.

Folic acid is a member of the B family of vitamins and plays an essential role in cell survival by participating in the biosynthesis of nucleic and amino acids. This essential vitamin is also a high affinity ligand that enhances the specificity of conjugated anti-cancer drugs by targeting folate receptor-positive cancer cells. It has been found that the folate receptor (FR) is up-regulated in more than 90% of non-mucinous ovarian carcinomas. The folate receptor is also found at high to moderate levels in kidney, brain, lung, and breast carcinomas. At the same time, it has been reported that the folate receptor occurs at low levels in most normal tissues leading to a mechanism for selectively targeting the cancer cells. Although the folate receptor can be used to deliver agents to tumor tissue with very high specificity, there are a number of cancers that do not express the folate receptor at all, or in sufficient numbers to provide the desired specificity. Thus, there is a need for developing targeted therapies to deliver agents to such folate receptor negative cancers.

Tumor-associated macrophages (TAMs) exist that are pro-tumorigenic. These macrophages are found in the tumor microenvironment, and can be pro-tumorigenic by causing such responses as inhibition of B and T cell activation, inhibition of tumor-associated antigen presentation, inhibition of cytotoxic granule release, and increased angiogenesis. Thus, therapies that deplete TAMs or inhibit their activity would be useful.

Applicants have discovered that tumors and cancers that do not express the folate receptor in sufficient numbers, or at all, can yet be treated by targeting drugs to TAMs. Described herein are methods for treating cancers by targeting TAMs using folate receptor binding compounds as TAM-targeting agent. Applicants have discovered that a subset of TAMs that is pro-tumorigenic expresses the folate receptor β, also known as folate receptor 2. Thus, Applicants have discovered that these pro-tumorigenic TAMs can be targeted using folate as the targeting ligand to deliver drugs to these TAMs to deplete or inhibit the pro-tumorigenic TAMs to treat cancer in a host animal whether or not the cancer cells themselves are targeted. It is to be understood that the methods described herein can be used to treat cancers that do not express the folate receptor, as well as cancers that do express the folate receptor.

In one embodiment, a method for treating a cancer is provided. The method comprises the steps of identifying the presence of tumor-associated macrophages in a cancer in a host animal, and administering to the host animal a therapeutically effective amount of a compound comprising a folate receptor binding compound attached to a drug via a linker.

In another embodiment, a method for treating a cancer is provided. The method comprises the step of administering to the host animal a therapeutically effective amount of a compound comprising a folate receptor binding compound attached to a drug via a linker wherein the host animal has previously been administered a folate imaging agent conjugate and the host animal's folate receptor status has been determined to be negative.

In another embodiment, a method for treating a cancer in a host animal by inhibiting or depleting tumor-associated macrophages in the host animal is provided. The method comprises the step of administering to the host animal a therapeutically effective amount of a compound comprising a folate receptor binding compound attached to a drug via a linker wherein the tumor-associated macrophages are inhibited or depleted.

In another embodiment, a method for targeting tumor-associated macrophages in a host animal is provided. The method comprises the step of administering to the host animal a therapeutically effective amount of a compound comprising a folate receptor binding compound attached to a drug via a linker wherein the tumor-associated macrophages are targeted.

In another embodiment, a method for treating a cancer in a host animal where tumor-associated macrophages are part of the cancer, tissue, or tumor is provided. The method comprises the steps of administering to the host animal a therapeutically effective amount of one or more compounds comprising a folate receptor binding compound attached to a drug via a linker, and treating the cancer having the tumor-associated macrophages. The folate in the one or more compounds is selected from the group consisting of a folate specific for the folate receptor-α and a folate specific for the folate receptor-β. In an alternative aspect, at least two compounds are administered and the folate in one compound is a folate specific for the folate receptor-α and the folate in the other compound is specific for the folate receptor-β.

In any of the embodiments described herein, the cancer may express folate receptors, or may not express folate receptors. In any of the embodiments in the preceding paragraphs, the folate can be specific for the folate receptor-β or the folate receptor-α. In any of the embodiments in the preceding paragraphs tumor associated macrophages are in the cancer and the tumor-associated macrophages may have the pro-tumor M2-biased CD163(+) phenotype, the pro-tumor M2-biased CD163(+) and TGF-β(+) phenotype, the pro-tumor M2-biased CD163(+), IL10(+), Arg1(+), TGF-β(+), VEGF(+), and CD206(+) phenotype, or the tumor-associated macrophages are pro-tumor M2-biased and may express one or more markers selected from the group consisting of CD163(+), IL10(+), Arg1(+), TGF-β(+), VEGF(+), and CD206(+).

In any of the embodiments described herein, the cancer can be selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis.

In any of the embodiments described herein, the drug can be of a class that is not an anti-mitotic drug, the drug can be selected from the group consisting of DNA-alkylating agents, trabectedin, doxorubicin, gemcitabine, bisphosphonates, and proapoptotic peptides, the drug can be selected from the group consisting of TLR9 agonists, TLR3 agonists, TLR7/8 agonists, monophosphoryl lipid A, mTOR inhibitors, PPARγ agonists, and PPARδ agonists, the drug can be a pyrrolobenzodiazepine (PBD), or the drug can be selected from the group consisting of silibinin, src kinase inhibitors, MerTK inhibitors, and Stat3 inhibitors. In any of these embodiments, the drug can inhibit the activity of the tumor-associated macrophages in the host animal. In any of these embodiments, the drug can deplete the tumor-associated macrophages in the host animal.

In any of the embodiments described herein, the compound, or a pharmaceutically acceptable salt thereof, can be administered to the host animal in a parenteral dosage form. The parenteral dosage form can be selected from the group consisting of intradermal, subcutaneous, intramuscular, intraperitoneal, intravenous, and intrathecal. In any of the embodiments described herein, the therapeutically effective amount can be from about 0.5 mg/m² to about 6.0 mg/m², from about 0.5 mg/m² to about 4.0 mg/m², or from about 0.5 mg/m² to about 2.0 mg/m².

It is appreciated herein that the presence of the tumor-associated macrophages in the tumor can indicate a poor prognosis for the host animal.

### BRIEF DESCRIPTION OF THE DRAWINGS

FIG. 1. Expression of FRβ (anti-mouse FRβ) on TAMs of breast cancer (MDA-MB-231 cells), non-small-cell lung cancer (A549 cells), mesothelioma (MSTO-211H cells), melanoma (B16/F10 cells), and Lewis lung carcinoma.

FIG. 2. Functional FRs are detected on both M1 and M2 F4/80(+) CD11b(+) TAMs in syngeneic mouse models (left bar=MHCIIhi F4/80(+) CD11b(+) TAMs [LLC tumor], M1; right bar=MHCIIlow F4/80(+) CD11b(+) TAMs [LLC tumor], M2).

FIG. 3. Functional FR levels on TAMs do not correlate with tumor weights.

FIG. 4. TAM density in nude rats bearing 4T1 mammary carcinoma correlates with tumor weight (≤1000 mg).

FIG. 5. FR-mediated uptake of a folate imaging agent in FR(−) 4T1 mammary tumors in nude rats.

FIG. 6. CD163(+) CD11b(+) 4T1 TAMs expressed a functional FR at ˜8-fold lower levels than KB cells. The first, second, and third items in the legend describe the leftmost, middle, and rightmost bars in each graph, respectively.

FIG. 7. Rat TG-macrophages express ˜2-fold higher functional FRβ than mouse TG-macrophages.

FIG. 8. Ex-vivo treatment showed selectivity of the folic acid drug conjugate Example 1 for FR(+) 4T1 TAMs over FR(−) 4T1 tumor cells.

FIG. 9. In vivo treatment showed a significant decrease in 4T1 TAMs in response to the folic acid drug conjugate Example 1.

FIG. 10. CD163(+) CD11b(+) 4T1 TAMs are most sensitive to the folic acid drug conjugate Example 1 induced early and late apoptosis. The first, second, and third items in the legend describe the leftmost, middle, and rightmost bars in each group in the graph, respectively. All images are from the same specimen (40×).

FIG. 11. IHC staining showed high FR-β expression in a human anaplastic thyroid cancer specimen.

FIG. 12. M2 macrophages are specifically depleted by clodronate liposomes.

FIG. 13. M2 macrophages are specifically depleted by clodronate liposomes.

### DETAILED DESCRIPTION OF ILLUSTRATIVE EMBODIMENTS

It is to be understood that each embodiment of the invention described herein may be, as applicable, combined with any other embodiment described herein. For example, any of the embodiments in the Summary, and/or of the enumerated clauses described herein, or any combination thereof, may be combined with any of the embodiments described in the Detailed Description.

Applicants have discovered methods for treating cancers by targeting TAMs (for example, pro-tumor M2-biased TAMs) using folate receptor binding compounds as TAM-targeting agents. Applicants have discovered that a subset of TAMs that is pro-tumorigenic expresses the folate receptor β which is useful for targeting TAMs with drugs using folates as targeting agents. In one embodiment, targeting of the pro-tumorigenic TAMs to deplete TAMs or to inhibit the activity of TAMs can result in inhibition of tumor growth, elimination of a tumor, or stable disease, and like therapeutic effects for the host animal. The methods described herein can be used to treat cancers that do not express the folate receptor, as well as cancers that do express the folate receptor.

In one embodiment, the tumor-associated macrophages described herein are pro-tumor and M2-biased, and, if depleted or inhibited, the host animal's condition may be improved. Such TAMs have a phenotype resulting from the expression of one or more markers selected from CD163(+), IL10(+), Arg1(+), TGF-β(+), VEGF(+), CD206(+), and combinations thereof. In another illustrative aspect, the tumor-associated macrophages described herein that are pro-tumor and M2-biased have a CD163(+) phenotype. In yet another embodiment, the tumor-associated macrophages described herein that are pro-tumor and M2-biased have a CD163(+) and TGF-β(+) phenotype. In another aspect, the tumor-associated macrophages described herein that are pro-tumor and M2-biased have a phenotype resulting from the expression of CD163(+), IL10(+), Arg1(+), TGF-β(+), VEGF(+), and CD206(+) markers. In another embodiment, the tumor-associated macrophages described herein that are pro-tumor and M2-biased have a phenotype resulting from the expression of one or more markers selected from the group consisting of CD163(+), IL10(+), Arg1(+), TGF-β(+), VEGF(+), and CD206(+). In one aspect, the presence of the tumor-associated macrophages (e.g., pro-tumor M2-biased TAMs) in the tumor indicates a poor prognosis for the host animal.

In one embodiment of the methods described herein for treating a cancer by targeting TAMs, the method comprises the steps of identifying the presence of tumor-associated macrophages (e.g., pro-tumor M2-biased TAMs) in a cancer in a host animal, and administering to the host animal a therapeutically effective amount of a compound comprising a folate attached to a drug via a linker.

In another embodiment, a method for treating a cancer by targeting TAMs (e.g., pro-tumor M2-biased TAMs) is provided. The method comprises the step of administering to the host animal a therapeutically effective amount of a compound comprising a folate attached to a drug via a linker wherein the host animal has previously been administered a folate imaging agent conjugate and the host animal's folate receptor status has been determined to be negative.

In yet another embodiment, a method for treating a cancer in a host animal by inhibiting or depleting tumor-associated macrophages (e.g., pro-tumor M2-biased TAMs) in the host animal is provided. The method comprises the step of administering to the host animal a therapeutically effective amount of a compound comprising a folate attached to a drug via a linker wherein the tumor-associated macrophages are inhibited or depleted.

In another aspect, a method of targeting tumor-associated macrophages (e.g., pro-tumor M2-biased TAMs) in a host animal is provided. The method comprises the step of administering to the host animal a therapeutically effective amount of a compound comprising a folate attached to a drug via a linker wherein the tumor-associated macrophages are targeted.

In yet another illustrative aspect, a method for treating a cancer in a host animal wherein tumor-associated macrophages are in the cancer is provided. The method comprises the steps of administering to the host animal a therapeutically effective amount of one or more compounds comprising a folate attached to a drug via a linker, and treating the cancer having the tumor-associated macrophages (e.g., pro-tumor M2-biased TAMs). In this illustrative aspect, the folate in the one or more compounds is selected from the group consisting of a folate specific for the folate receptor-α and a folate specific for the folate receptor-β. In an alternative aspect, at least two compounds are administered and the folate in one compound is a folate specific for the folate receptor-α and the folate in the other compound is specific for the folate receptor-β.

In any method embodiment described herein, the folate can be a folate specific for the folate receptor-α or a folate specific for the folate receptor-β. The phrase “wherein tumor-associated macrophages are in the cancer” used herein generally refers to the tumor associated macrophages (e.g., pro-tumor M2-biased TAMs) that exist in the microenvironment of a cancer (e.g., a tumor), or, for example, are found in cancerous tissue (e.g., tumor tissue).

The methods described herein are used to treat a “host animal” with cancer in need of such treatment. In one embodiment, the methods described herein can be used for both human clinical medicine and veterinary applications. Thus, a “host animal” can be administered the compounds or folate-imaging agent conjugates described herein (described below), and the host animal can be human or, in the case of veterinary applications, can be a laboratory, agricultural, domestic, or wild animal. In one aspect, the host animal can be a human, a laboratory animal such as a rodent (e.g., mice, rats, hamsters, etc.), a rabbit, a monkey, a chimpanzee, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In various embodiments, the cancers described herein can be a cancer cell population that is tumorigenic, including benign tumors and malignant tumors, or the cancer can be non-tumorigenic. The cancer can arise spontaneously or by such processes as mutations present in the germline of the host animal or by somatic mutations, or the cancer can be chemically-, virally-, or radiation-induced. Cancers applicable to the invention described herein include, but are not limited to, a carcinoma, a sarcoma, a lymphoma, a melanoma, a mesothelioma, a nasopharyngeal carcinoma, a leukemia, an adenocarcinoma, and a myeloma.

In some aspects the cancer can be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, prostate cancer, leukemia, lymphoma, pleural mesothelioma, cancer of the bladder, Burkitt's lymphoma, cancer of the ureter, cancer of the kidney, neoplasms of the central nervous system, brain cancer, pituitary adenoma, or adenocarcinoma of the gastroesophageal junction.

In some aspects the cancers can be selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis. Any cancer that has tumor-associated macrophages (e.g., pro-tumor M2-biased TAMs) can be treated in accordance with the invention.

Folate receptor binding compounds and ligands that can be used to form the conjugates described herein are generally described in U.S. Published Application No. 2013/0116195, the disclosure of which is incorporated herein by reference.

Any drug suitable for depleting or inhibiting TAMs (e.g., pro-tumor M2-biased TAMs) can be used in accordance with the invention. TAMs are not highly proliferative. Thus, in one embodiment, the drug is not an anti-mitotic drug. In another illustrative aspect, the drug can be selected from the group consisting of a DNA-alkylating agent, trabectedin, doxorubicin, gemcitabine, a bisphosphonate (e.g., free or in liposomal form), and a proapoptotic peptide. In another illustrative aspect, the drug can be a proapoptotic peptide (e.g., with a sequence of KLAKLAKKLAKLAK (SEQ ID NO: 1)).

In yet another embodiment, the drug can be selected from the group consisting of a TLR9 agonist, a TLR3 agonist, a TLR7/8 agonist, a monophosphoryl lipid A (e.g., detoxified LPS), an mTOR inhibitor, a PPARγ agonist, and a PPARδ agonist. In one illustrative aspect, the drug is a TLR9 agonist (e.g., a CpG oliogdeoxynucleotide). In another embodiment, the drug is a TLR3 agonist (e.g., Poly: IC). In yet another embodiment, the drug is a TLR7/8 agonist (e.g., imiquimod). In another embodiment, the drug is an mTOR inhibitor (e.g., an everolimus or a rapamycin).

In another aspect, the drug can be selected from the group consisting of silibinin, a src kinase inhibitor, a MerTK inhibitor, and a Stat3 inhibitor. In this embodiment, the drug can be a src kinase inhibitor (e.g., dasatinib). In another embodiment, the drug can be a MerTK inhibitor (e.g., UNC1062). In yet another embodiment, the drug can be a Stat3 inhibitor (e.g., selected from sunitinib and sorafenib).

In another embodiment, the drug is a pyrrolobenzodiazepine (PBD).

It is to be understood that analogs or derivatives of the drugs described herein may also be used in the compounds, compositions, and methods described herein.

In another embodiment, the compound has the formula

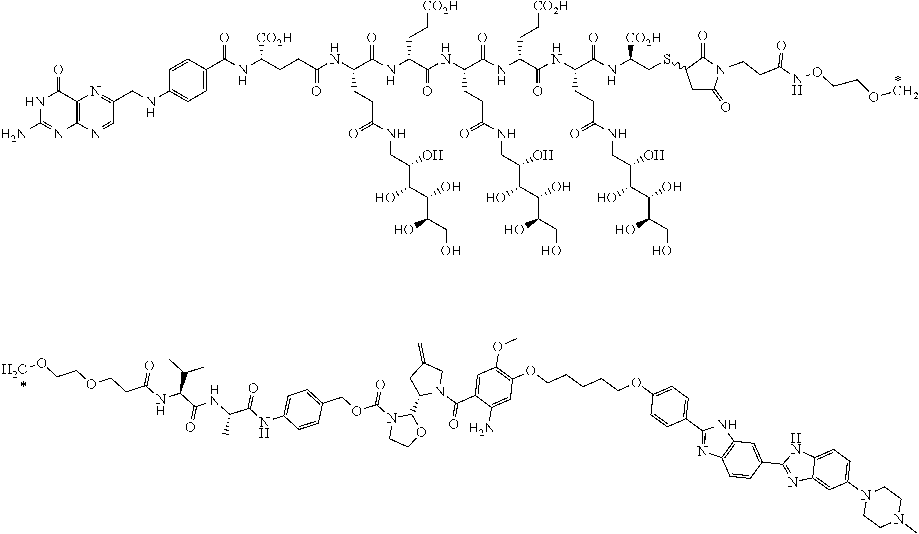

or a pharmaceutically acceptable salt thereof, wherein the carbons labeled with * are connected by a covalent bond.

Additional illustrative and non-limiting embodiments of the invention are described in the following enumerated clauses. All combinations of the following clauses are understood to be additional embodiments of the invention described herein.

1. A method for treating a cancer comprising the steps of identifying the presence of tumor-associated macrophages in the cancer in a host animal, and administering to the host animal a therapeutically effective amount of one or more compounds comprising a folate receptor binding compound attached to a drug via a linker.

2. A method for treating a cancer in a host animal, the method comprising the step of administering to the host animal a therapeutically effective amount of one or more compounds comprising a folate receptor binding compound attached to a drug via a linker to inhibit or deplete tumor-associated macrophages in the host animal.

3. A method for targeting tumor-associated macrophages in a host animal, the method comprising the step of administering to the host animal a therapeutically or diagnostically effective amount of one or more compounds comprising a folate receptor binding compound attached to a drug via a linker to target the tumor-associated macrophages.

4. The method of any one of clauses 1 to 3 wherein the folate receptor binding compound is specific for the folate receptor-n also referred to as folate receptor 2.

5. A method for treating a cancer in a host animal where tumor-associated macrophages are in the cancer and/or form part of the tissue or tumor, the method comprising the steps of administering to the host animal a therapeutically effective amount of one or more compounds comprising a folate receptor binding compound attached to a drug via a linker, and treating the cancer having the tumor-associated macrophages.

6. The method of any one of clauses 1 to 5 wherein tumor associated macrophages are in the cancer and the tumor-associated macrophages have the pro-tumor M2-biased CD163(+) phenotype.

7. The method of any one of clauses 1 to 6 wherein tumor-associated macrophages are in the cancer and the tumor-associated macrophages have the pro-tumor M2-biased CD163(+) and TGF-β(+) phenotype.

8. The method of any one of clauses 1 to 7 wherein tumor-associated macrophages are in the cancer and the tumor-associated macrophages have the pro-tumor M2-biased CD163(+), IL10(+), Arg1(+), TGF-β(+), VEGF(+), and CD206(+) phenotype.

9. The method of any one of clauses 1 to 8 wherein the tumor-associated macrophages are in the cancer and the tumor-associated macrophages are pro-tumor M2-biased and express one or more markers selected from the group consisting of CD163(+), IL10(+), Arg1(+), TGF-β(+), VEGF(+), and CD206(+).

10. The method of any one of clauses 1 to 9 wherein tumor-associated macrophages are in the cancer and/or form part of the tissue or tumor and the tumor-associated macrophages are pro-tumor M2-biased and express one or more markers selected from the group consisting of CD163(+), IL10(+), Arg1(+), TGF-β(+), VEGF(+), and CD206(+).

11. The method of any one of clauses 1 to 11 wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis.

12. The method of any one of clauses 1 to 11 wherein the drug is not an anti-mitotic drug.

13. The method of any one of clauses 1 to 12 wherein the drug is selected from the group consisting of DNA-alkylating agents, pyrrolobenzodiazepines (PBDs), trabectedin, doxorubicin, gemcitabine, bisphosphonates, and proapoptotic peptides.

14. The method of any one of the clauses 1 to 13 wherein the drug is doxorubicin.

15. The method of any one of the clauses 1 to 13 wherein the drug is a proapoptotic peptide.

16. The method of clause 13 or 15 wherein the proapoptotic peptide has a sequence consisting of SEQ ID NO: 1.

17. The method of any one of clauses 1 to 11 wherein the drug is selected from the group consisting of TLR9 agonists, TLR3 agonists, TLR7/8 agonists, a monophosphoryl lipid A, mTOR inhibitors, PPARγ agonists, and PPARδ agonists.

18. The method of any one of clauses 1 to 11 or 17 wherein the drug is a TLR9 agonist.

19. The method of any one of clauses 1 to 11 wherein the drug is a CpG oliogdeoxynucleotide.

20. The method of any one of clauses 1 to 11 or 17 wherein the drug is a TLR3 agonist.

21. The method of any one of clauses 1 to 11 wherein the drug is polyinosinic:polycytidylic acid (poly I:C).

22. The method of any one of clauses 1 to 11 or 17 wherein the drug is a TLR7/8 agonist.

23. The method of any one of clauses 1 to 11 or 17 wherein the drug is an mTOR inhibitor.

24. The method of any one of clauses 1 to 11 or 17 wherein the drug is an everolimus or a rapamycin.

25. The method of any one of clauses 1 to 11 wherein the drug is imiquimod.

26. The method of any one of clauses 1 to 11 wherein the drug is selected from the group consisting of silibinin, src kinase inhibitors, MerTK inhibitors, and Stat3 inhibitors.

27. The method of any one of clauses 1 to 11 or 26 wherein the drug is a src kinase inhibitor.

28. The method of any one of clauses 1 to 11 or 27 wherein the drug is dasatinib.

29. The method of any one of clauses 1 to 11 or 26 wherein the drug is a MerTK inhibitor.

30. The method of any one of clauses 1 to 11 or 29 wherein the drug is UNC1062.

31. The method of any one of clauses 1 to 11 or 26 wherein the drug is a Stat3 inhibitor.

32. The method of any one of clauses 1 to 11, 26, or 31 wherein the drug is sunitinib.

33. The method of any one of clauses 1 to 11, 26, or 31 wherein the drug is sorafenib.

34. The method of any one of clauses 1 to 11 or 13 wherein at least one of the compounds has the formula

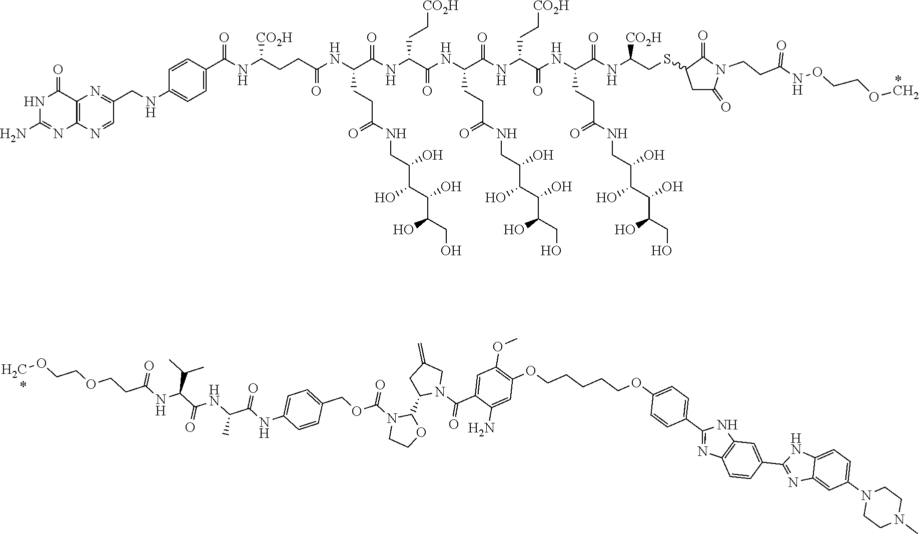

or a pharmaceutically acceptable salt thereof, wherein the carbons labeled with * are connected by a covalent bond.

35. The method of any one of clauses 1 to 34 wherein the drug is capable of depleting, or depletes the tumor-associated macrophages in the host animal.

36. The method of any one of clauses 1 to 34 wherein the drug is capable of inhibiting, or inhibits the activity of the tumor-associated macrophages in the host animal.

37. The method of any one of clauses 1 to 36 wherein the compound, or a pharmaceutically acceptable salt thereof, is administered to the host animal in a parenteral dosage form.

38. The method of clause 37 wherein the parenteral dosage form is selected from the group consisting of intradermal, subcutaneous, intramuscular, intraperitoneal, intravenous, and intrathecal.

39. The method of any one of clauses 1 to 38 wherein the therapeutically effective amount is from about 0.5 mg/m² to about 6.0 mg/m².

40. The method of any one of clauses 1 to 39 wherein the therapeutically effective amount is from about 0.5 mg/m² to about 4.0 mg/m².

41. The method of any one of clauses 1 to 40 wherein the therapeutically effective amount is from about 0.5 mg/m² to about 2.0 mg/m².

The compounds described herein may contain one or more chiral centers, or may otherwise be capable of existing as multiple stereoisomers. It is to be understood that in one embodiment, the invention described herein is not limited to any particular stereochemical requirement, and that the compounds may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configurations at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “tumor associated macrophages” (TAMs) generally refers to macrophages that exist in the microenvironment of a cancer, for example, a tumor.

As used herein, the term “inhibiting tumor associated macrophages” generally refers to reducing the activity or eliminating the activity of TAMs, such as by reducing or eliminating the ability of TAMs to stimulate angiogenesis in tumor tissue.

As used herein, the term “depleting tumor associated macrophages” generally refers to reducing the number of TAMs, eliminating TAMs, or repolarizing TAMs, including causing TAMs to shift from an M2 to an M1 phenotype.

As used herein, the term “pro-tumor” with reference to TAMs generally refers to TAMs that enhance tumorgenesis, such as, for example, by inhibiting B and/or T cell activation, inhibiting tumor-associated antigen presentation, inhibiting cytotoxic granule release, and/or increasing angiogenesis.

As used herein, the term “M2-biased” generally refers to TAMs that are pro-tumor TAMs.

As used herein, the term “administering” generally refers to any and all means of introducing compounds described herein to the host animal, including, but not limited to, by oral (po), intravenous (iv), intramuscular (im), subcutaneous (sc), transdermal, inhalation, buccal, ocular, sublingual, vaginal, rectal, and like routes of administration. Compounds described herein may be administered in unit dosage forms and/or formulations containing one or more pharmaceutically-acceptable carriers, adjuvants, diluents, excipients, and/or vehicles, and combinations thereof.

As used herein, the term “composition” generally refers to any product comprising more than one ingredient. It is to be understood that the compositions described herein may be prepared from isolated compounds described herein or from salts, solutions, hydrates, solvates, and other forms of the compounds described herein. It is appreciated that certain functional groups, such as the hydroxy, amino, and like groups may form complexes with water and/or various solvents, in the various physical forms of the compounds. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein.

As used herein, the term “linker” includes a chain of atoms that connects two or more functional parts of a molecule to form a compound of the invention. Illustratively, the chain of atoms is selected from C, N, O, S, Si, and P, or C, N, O, S, and P, C, N, O, and S. The chain of atoms covalently connects different functional capabilities of the compound, such as the folate and the drug. The linker may have a wide variety of lengths, such as in the range from about 2 to about 100 atoms in the contiguous backbone. The atoms used in forming the linker may be combined in all chemically relevant ways, such as chains of carbon atoms forming alkylene, alkenylene, and alkynylene groups, and the like; chains of carbon and oxygen atoms forming ethers, polyoxyalkylene groups, or when combined with carbonyl groups forming esters and carbonates, and the like; chains of carbon and nitrogen atoms forming amines, imines, polyamines, hydrazines, hydrazones, or when combined with carbonyl groups forming amides, ureas, semicarbazides, carbazides, and the like; chains of carbon, nitrogen, and oxygen atoms forning alkoxyamines, alkoxylamines, or when combined with carbonyl groups forming urethanes, amino acids, acyloxylamines, hydroxamic acids, and the like; and many others. In addition, it is to be understood that the atoms forming the chain in each of the foregoing illustrative embodiments may be either saturated or unsaturated, thus forming single, double, or triple bonds, such that for example, alkanes, alkenes, alkynes, imines, and the like may be radicals that are included in the linker. In addition, it is to be understood that the atoms forming the linker may also be cyclized upon each other or be part of cyclic structures to form divalent cyclic structures that form the linker, including cyclo alkanes, cyclic ethers, cyclic amines, and other heterocycles, arylenes, heteroarylenes, and the like in the linker. In this latter arrangement, it is to be understood that the linker length may be defined by any pathway through the one or more cyclic structures. Illustratively, the linker length is defined by the shortest pathway through the each one of the cyclic structures. It is to be understood that the linkers may be optionally substituted at any one or more of the open valences along the chain of atoms, such as optional substituents on any of the carbon, nitrogen, silicon, or phosphorus atoms. It is also to be understood that the linker may connect the two or more functional parts of a molecule to form a compound at any open valence, and it is not necessary that any of the two or more functional parts of a molecule forming the compound are attached at any apparent end of the linker.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is to be further understood that in certain embodiments, alkenyl and/or alkynyl may each be advantageously of limited length, including C₂-C₂₄, C₂-C₁₂, C₂-C₈, C₂-C₆, and C₂-C₄. Illustratively, such particularly limited length alkenyl and/or alkynyl groups, including C₂-C₈, C₂-C₆, and C₂-C₄ may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄. Illustratively, such particularly limited length alkylene groups, including C₁-C₈, C₁-C₆, and C₁-C₄ may be referred to as lower alkylene. It is to be further understood that in certain embodiments, alkenylene and/or alkynylene may each be advantageously of limited length, including C₂-C₂₄, C₂-C₁₂, C₂-C₈, C₂-C₆, and C₂-C₄. Illustratively, such particularly limited length alkenylene and/or alkynylene groups, including C₂-C₈, C₂-C₆, and C₂-C₄ may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain is cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “carboxylic acid and derivatives thereof” includes the group CO₂H and salts thereof, and esters and amides thereof, and CN.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups, also referred to herein as aryl subsituents, illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein, the term amino acid refers generally to beta, gamma, and longer amino acids, such as amino acids of the formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like.

In one embodiment, the linker (L) described herein may include one or more hydrophilic portions. It is appreciated that the arrangement and/or orientation of the various hydrophilic linker portions may be in a linear or branched fashion, or both. For example, the hydrophilic linker portions may form the backbone of the linker forming the compound. Alternatively, the hydrophilic portion of the linker may be pendant to or attached to the backbone of the chain of atoms connecting the folate to the drug. In this latter arrangement, the hydrophilic portion may be proximal or distal to the backbone chain of atoms.

In another embodiment, the linker is more or less linear, and the hydrophilic groups are arranged largely in a series to form a chain-like linker in the compound. Said another way, the hydrophilic groups form some or all of the backbone of the linker in this linear embodiment.

In another embodiment, the linker is branched with hydrophilic groups. In this branched embodiment, the hydrophilic groups may be proximal to the backbone or distal to the backbone. In each of these arrangements, the linker is more spherical or cylindrical in shape. In one variation, the linker is shaped like a bottle-brush. In one aspect, the backbone of the linker is formed by a linear series of amides, and the hydrophilic portion of the linker is formed by a parallel arrangement of branching side chains, such as by connecting monosaccharides, sulfonates, and the like, and derivatives and analogs thereof.

It is understood that the linker may be neutral or ionizable under certain conditions, such as physiological conditions encountered in vivo. For ionizable linkers, under the selected conditions, the linker may deprotonate to form a negative ion, or alternatively become protonated to form a positive ion. It is appreciated that more than one deprotonation or protonation event may occur. In addition, it is understood that the same linker may deprotonate and protonate to form inner salts or zwitterions.

In another embodiment, the hydrophilic linkers, or portions thereof, are neutral, i.e. under physiological conditions, the linkers do not significantly protonate nor deprotonate. In another embodiment, the hydrophilic linkers, or portions thereof, may be protonated to carry one or more positive charges. It is understood that the protonation capability is condition dependent. In one aspect, the conditions are physiological conditions, and the linker is protonated in vivo. In another embodiment, the linkers, or portions thereof, include both regions that are neutral and regions that may be protonated to carry one or more positive charges. In another embodiment, the linkers, or portions thereof, include both regions that may be deprotonated to carry one or more negative charges and regions that may be protonated to carry one or more positive charges. It is understood that in this latter embodiment that zwitterions or inner salts may be formed.

In one aspect, the regions of the linkers that may be deprotonated to carry a negative charge include carboxylic acids, such as aspartic acid, glutamic acid, and longer chain carboxylic acid groups, and sulfuric acid esters, such as alkyl esters of sulfuric acid. In another aspect, the regions of the linkers that may be protonated to carry a positive charge include amino groups, such as polyaminoalkylenes including ethylene diamines, propylene diamines, butylene diamines and the like, and/or heterocycles including pyrollidines, piperidines, piperazines, and other amino groups, each of which is optionally substituted. In another embodiment, the regions of the linkers that are neutral include poly hydroxyl groups, such as sugars, carbohydrates, saccharides, inositols, and the like, and/or polyether groups, such as polyoxyalkylene groups including polyoxyethylene, polyoxypropylene, and the like.

In one embodiment, the hydrophilic linkers, or portions thereof, described herein include are formed primarily from carbon, hydrogen, and oxygen, and have a carbon/oxygen ratio of about 3:1 or less, or of about 2:1 or less. In one aspect, the hydrophilic linkers described herein include a plurality of ether functional groups. In another aspect, the hydrophilic linkers described herein include a plurality of hydroxyl functional groups. Illustrative fragments that may be used to form such linkers include polyhydroxyl molecules such as carbohydrates, polyether molecules such as polyethylene glycol units, and acid groups such as carboxyl and alkyl sulfuric acids. In one variation, oligoamide groups, and the like may also be included in the linker.

Illustrative carbohydrate linker portions include saccharopeptides as described herein that include both a peptide feature and sugar feature; glucuronides, which may be incorporated via [2+3] Huisgen cyclization, also known as click chemistry; j-alkyl glycosides, such as of 2-deoxyhexapyranoses (2-deoxyglucose, 2-deoxyglucuronide, and the like), and β-alkyl mannopyranosides. Illustrative PEG groups include those of a specific length range from about 4 to about 20 PEG groups. Illustrative alkyl sulfuric acid esters may also be introduced with click chemistry directly into the backbone. Illustrative oligoamide linker portions include EDTA and DTPA, n-amino acids, and the like.

In another embodiment, the hydrophilic linkers, or portions thereof, described herein include a polyether, according to the following formulae:

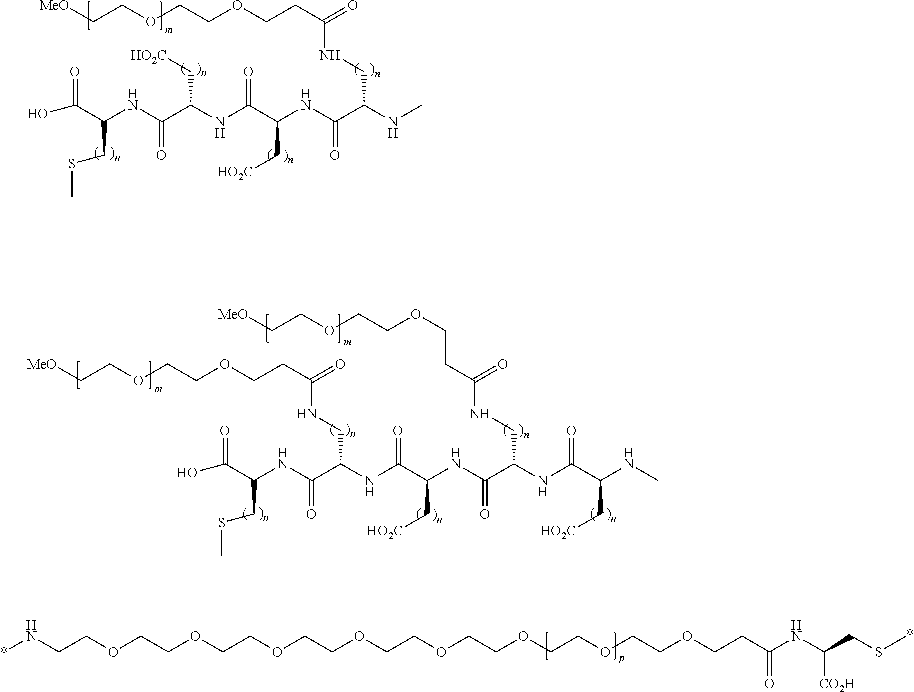

where m is an integer independently selected in each instance from 1 to about 8; p is an integer selected 1 to about 10; and n is an integer independently selected in each instance from 1 to about 3. In one aspect, m is independently in each instance 1 to about 3. In another aspect, n is 1 in each instance. In another aspect, p is independently in each instance about 4 to about 6. Illustratively, the corresponding polypropylene polyethers corresponding to the foregoing are described herein and may be included in the compounds as hydrophilic linkers, or portions thereof. In addition, it is appreciated that mixed polyethylene and polypropylene polyethers may be included in the compounds as hydrophilic linkers, or portions thereof. Further, cyclic variations of the foregoing polyethers, such as those that include tetrahydrofuranyl, 1,3-dioxanes, 1,4-dioxanes, and the like are described herein.

In another illustrative embodiment, the hydrophilic linkers, or portions thereof, described herein include a plurality of hydroxyl functional groups, such as linkers that incorporate monosaccharides, oligosaccharides, polysaccharides, and the like. It is to be understood that the polyhydroxyl containing linker portions comprise a plurality of —(CROH)— groups, where R is hydrogen or alkyl.

In another embodiment, the linkers, or portions thereof, include one or more of the following fragments:

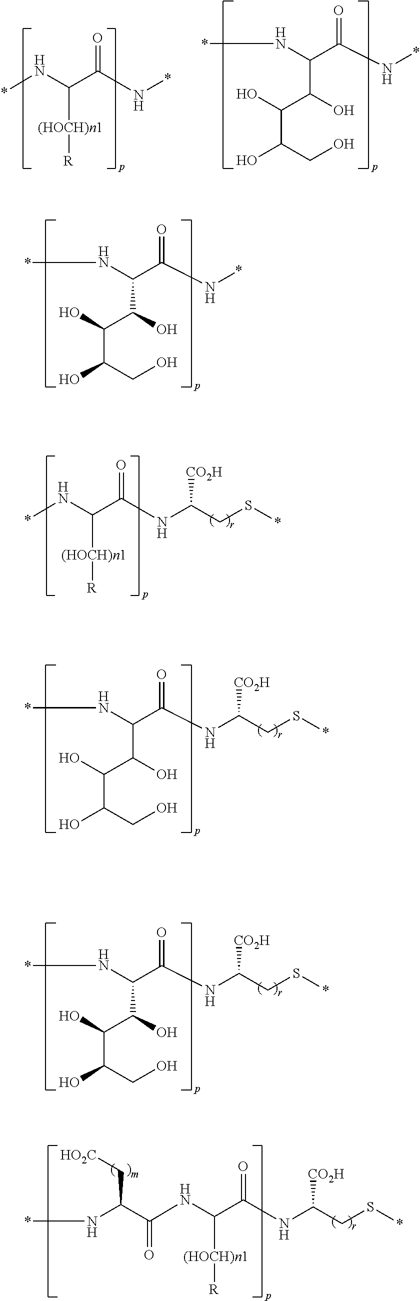

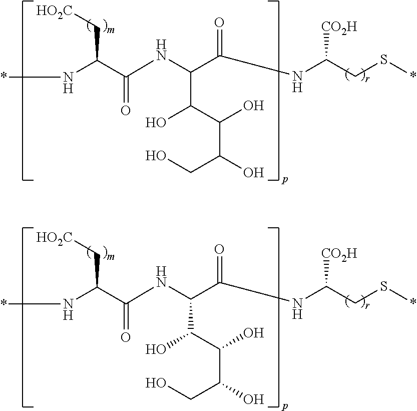

wherein R is H, alkyl, cycloalkyl, or arylalkyl; m is an integer from 1 to about 3; n1 is an integer from 1 to about 5, or n1 is an integer from 2 to about 5, p is an integer from 1 to about 5, and r is an integer selected from 1 to about 3. In one aspect, the integer n is 3 or 4. In another aspect, the integer p is 3 or 4. In another aspect, the integer r is 1.

In another embodiment, the linkers, or portions thereof, include one or more of the following fragments:

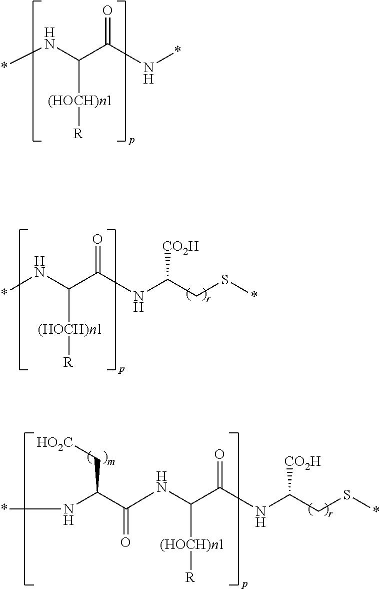

wherein R is H, alkyl, cycloalkyl, or arylalkyl; m is an integer from 1 to about 3; n1 is an integer from 1 to about 5, or from 2 to about 5, p is an integer from 1 to about 5, and r is an integer selected from 1 to about 3. In one aspect, the integer n is 3 or 4. In another aspect, the integer p is 3 or 4. In another aspect, the integer r is 1.

In another embodiment, the linkers, or portions thereof, include one or more of the following cyclic polyhydroxyl groups:

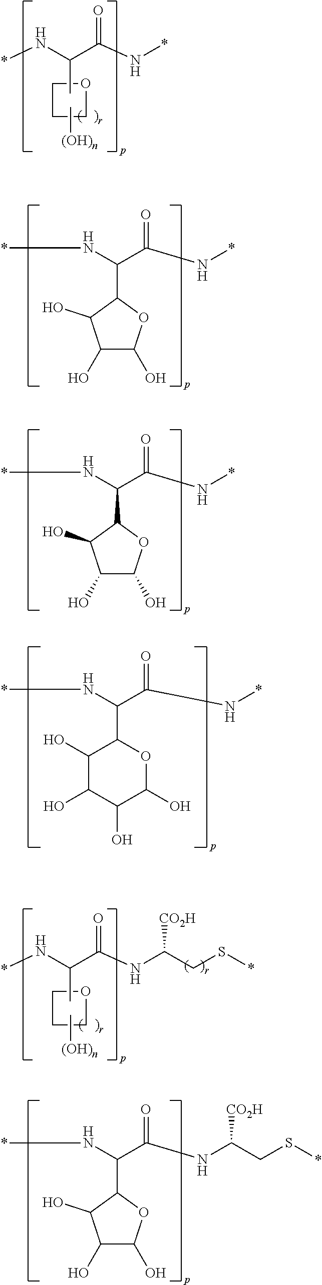

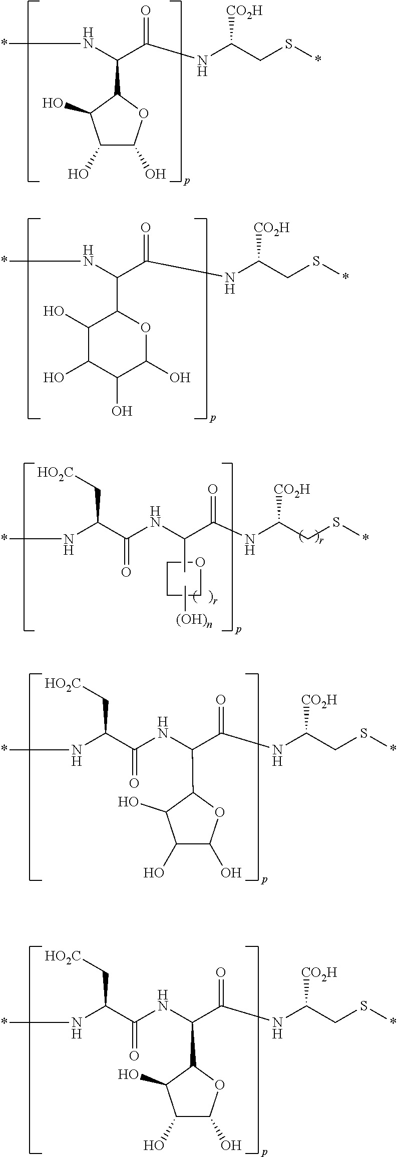

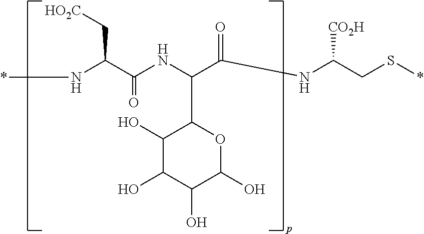

wherein n is an integer from 2 to about 5, p is an integer from 1 to about 5, and r is an integer from 1 to about 4. In one aspect, the integer n is 3 or 4. In another aspect, the integer p is 3 or 4. In another aspect, the integer r is 2 or 3. It is understood that all stereochemical forms of such sections of the linkers are described herein. For example, in the above formulae, the section may be derived from ribose, xylose, glucose, mannose, galactose, or other sugar and retain the stereochemical arrangements of pendant hydroxyl and alkyl groups present on those molecules. In addition, it is to be understood that in the foregoing formulae, various deoxy groups are also described. Illustratively, groups of the following formulae are described:

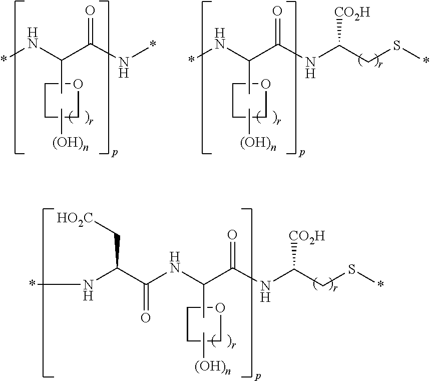

wherein n is equal to or less than r, such as when r is 2 or 3, n is 1 or 2, or 1, 2, or 3, respectively.

In another embodiment, the linker, or portion thereof, includes a polyhydroxyl of the following formula:

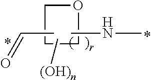

wherein n and r are each an integer selected from 1 to about 3. In one aspect, the linker, or portion thereof, includes one or more polyhydroxyls of the following formulae:

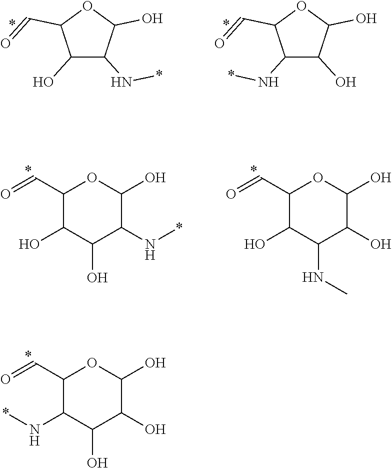

It is understood that all stereochemical forms of such sections of the linkers are described herein. For example, in the above formula, the section may be derived from ribose, xylose, glucose, mannose, galactose, or other sugar and retain the stereochemical arrangements of pendant hydroxyl and alkyl groups present on those molecules.

In another configuration, the hydrophilic linkers described herein include polyhydroxyl groups that are spaced away from the backbone of the linker. In one embodiment, such carbohydrate groups or polyhydroxyl groups are connected to the backbone by a triazole group, forming triazole-linked hydrophilic linkers, or portions thereof. Illustratively, such linkers include fragments of the following formulae:

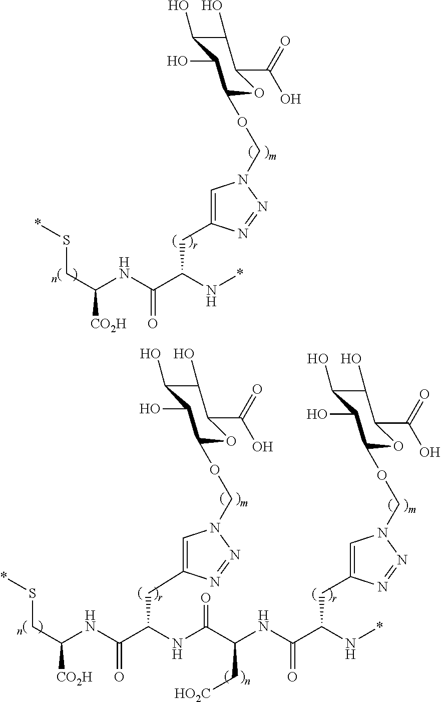

wherein n, m, and r are integers and are each independently selected in each instance from 1 to about 5. In one illustrative aspect, m is independently 2 or 3 in each instance. In another aspect, r is 1 in each instance. In another aspect, n is 1 in each instance. In one variation, the group connecting the polyhydroxyl group to the backbone of the linker is a different heteroaryl group, including but not limited to, pyrrole, pyrazole, 1,2,4-triazole, furan, oxazole, isoxazole, thienyl, thiazole, isothiazole, oxadiazole, and the like. Similarly, divalent 6-membered ring heteroaryl groups are described. Other variations of the foregoing illustrative hydrophilic linkers, or portions thereof, include oxyalkylene groups, such as the following formulae:

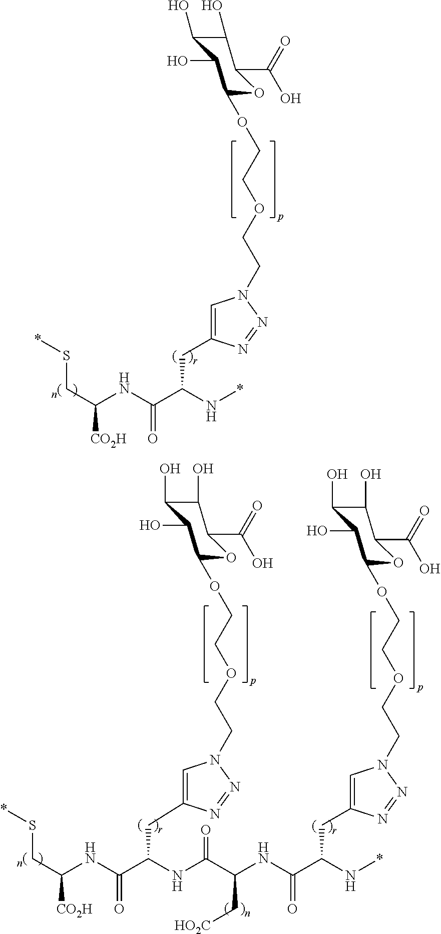

wherein n and r are integers and are each independently selected in each instance from 1 to about 5; and p is an integer selected from 1 to about 4.

In another embodiment, such carbohydrate groups or polyhydroxyl groups are connected to the backbone by an amide group, forming amide-linked hydrophilic linkers, or portions thereof. Illustratively, such linkers include fragments of the following formulae:

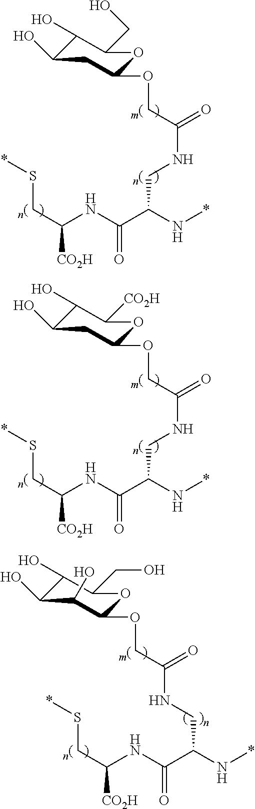

wherein n is an integer selected from 1 to about 3, and m is an integer selected from 1 to about 22. In one illustrative aspect, n is 1 or 2. In another illustrative aspect, m is selected from about 6 to about 10, illustratively 8. In one variation, the group connecting the polyhydroxyl group to the backbone of the linker is a different functional group, including but not limited to, esters, ureas, carbamates, acylhydrazones, and the like. Similarly, cyclic variations are described. Other variations of the foregoing illustrative hydrophilic linkers, or portions thereof, include oxyalkylene groups, such as the following formulae:

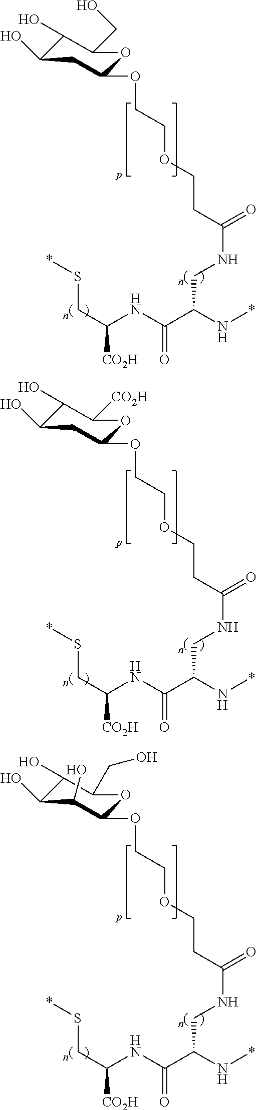

wherein n and r are integers and are each independently selected in each instance from 1 to about 5; and p is an integer selected from 1 to about 4.

In another embodiment, the linkers, or portions thereof, include one or more of the following fragments:

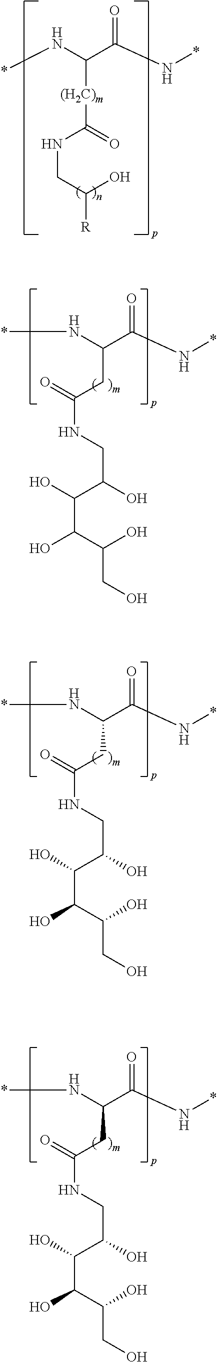

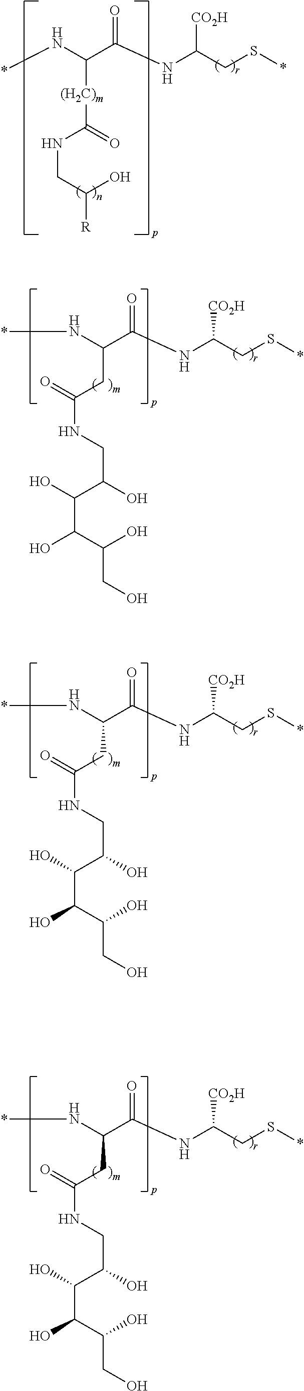

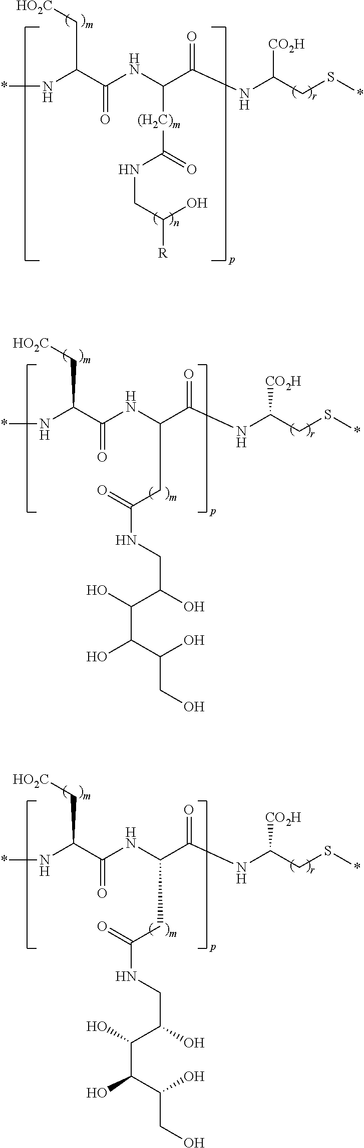

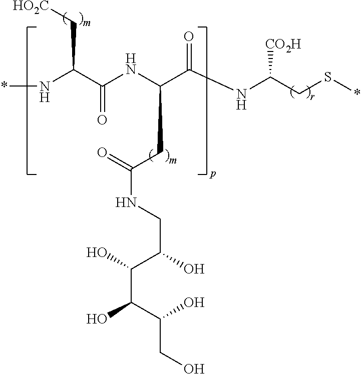

wherein R is H, alkyl, cycloalkyl, or arylalkyl; m is an independently selected integer from 1 to about 3; n is an integer from 1 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the linkers, or portions thereof, include one or more of the following fragments:

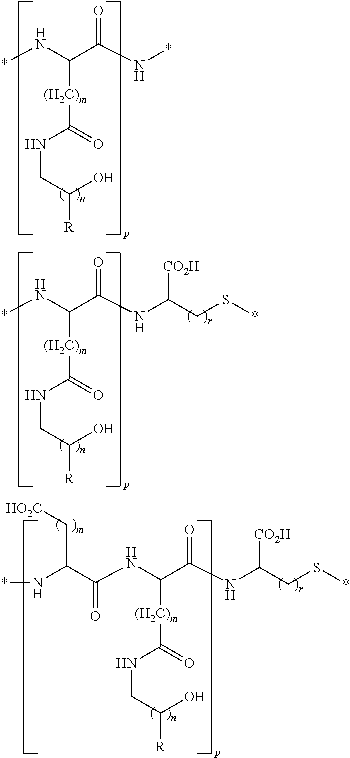

wherein R is H, alkyl, cycloalkyl, or arylalkyl; m is an independently selected integer from 1 to about 3; n is an integer from 2 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the linkers, or portions thereof, include one or more of the following fragments:

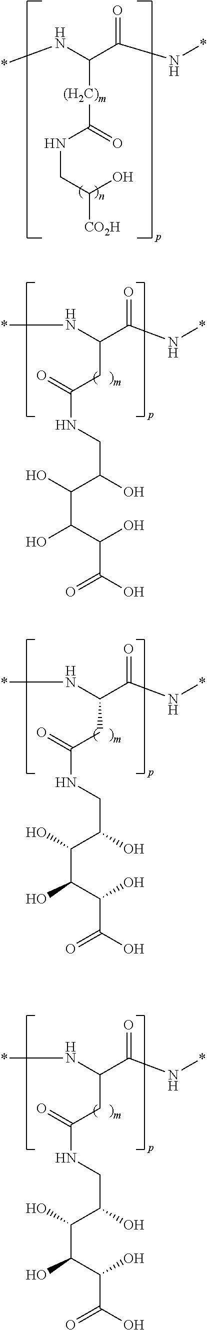

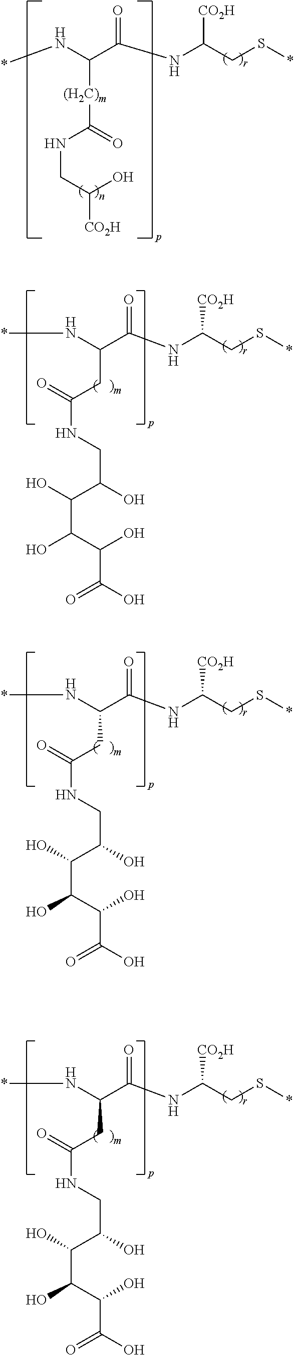

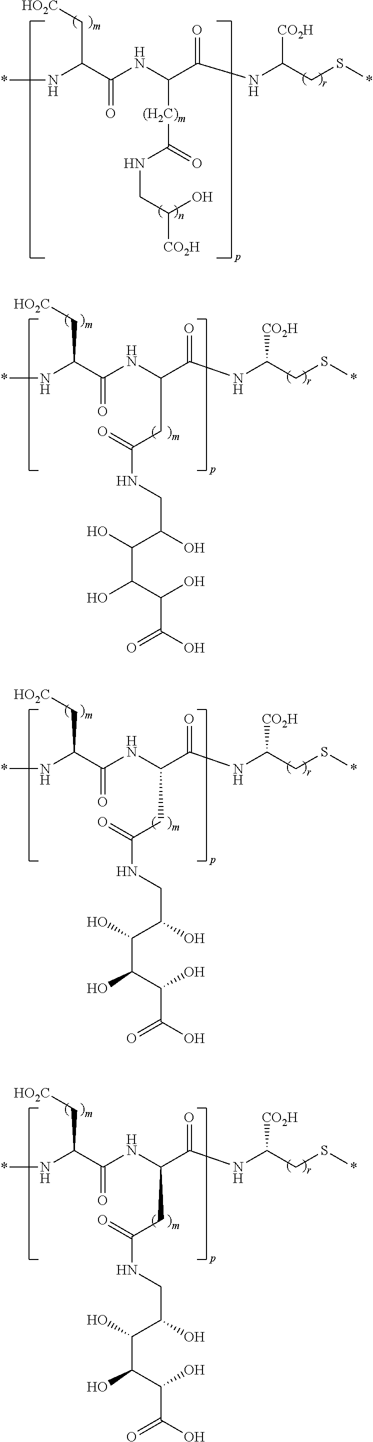

wherein m is an independently selected integer from 1 to about 3; n is an integer from 1 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the linkers, or portions thereof, include one or more of the following fragments:

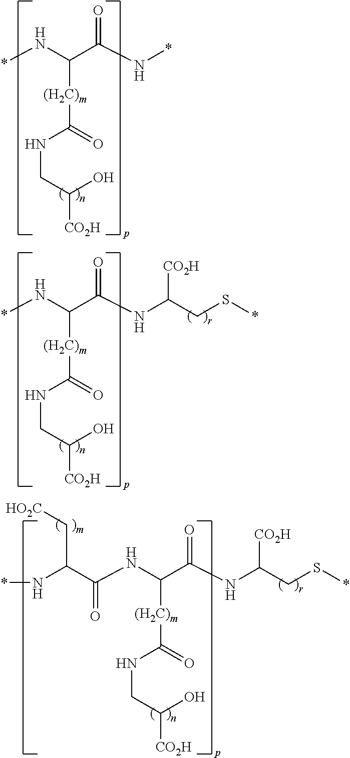

wherein m is an independently selected integer from 1 to about 3; n is an integer from 2 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the linkers, or portions thereof, include one or more of the following fragments:

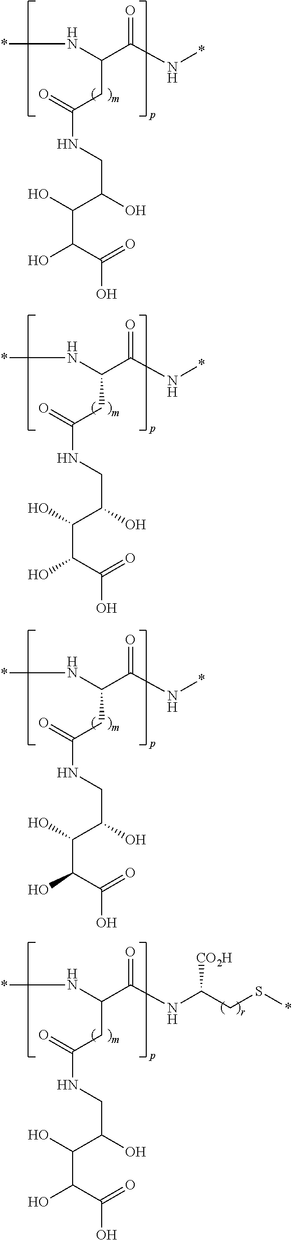

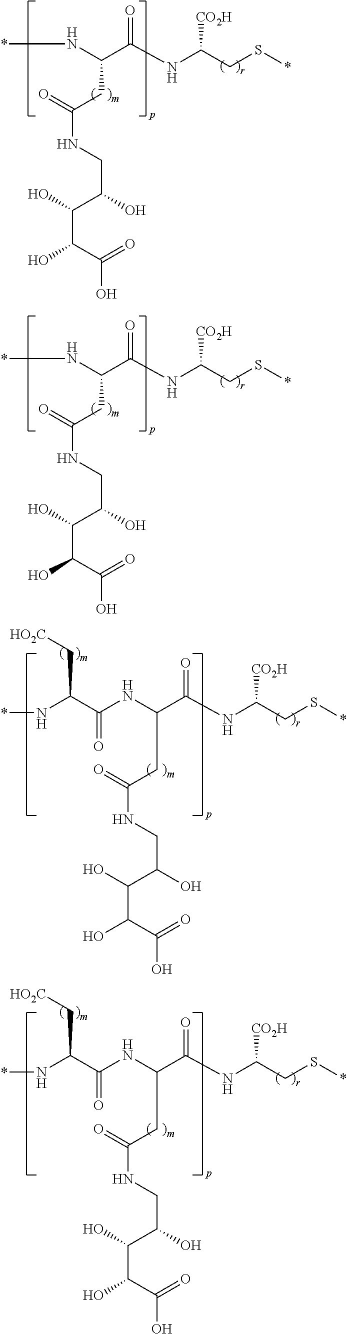

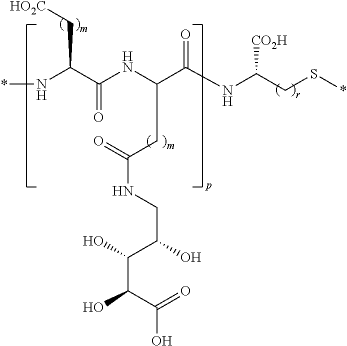

wherein m is an independently selected integer from 1 to about 3; n is an integer from 1 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the hydrophilic linker, or a portion thereof, is a combination of backbone and branching side motifs such as is illustrated by the following formulae

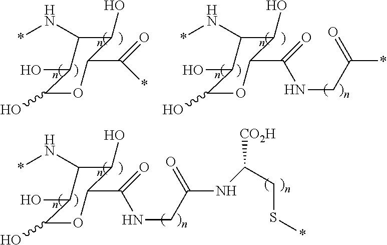

wherein n is an integer independently selected in each instance from 0 to about 3. The above formulae are intended to represent 4, 5, 6, and even larger membered cyclic sugars. In addition, it is to be understood that the above formulae may be modified to represent deoxy sugars, where one or more of the hydroxy groups present on the formulae are replaced by hydrogen, alkyl, or amino. In addition, it is to be understood that the corresponding carbonyls are described by the above formulae, where one or more of the hydroxyl groups is oxidized to the corresponding carbonyl. In addition, in this illustrative embodiment, the pyranose includes both carboxyl and amino functional groups which (a) can be inserted into the backbone and (b) can provide synthetic handles for branching side chains in variations of this embodiment. Any of the pendant hydroxyl groups may be used to attach other chemical fragments, including additional sugars to prepare the corresponding oligosaccharides. Other variations of this embodiment are also described, including inserting the pyranose or other sugar into the backbone at a single carbon, i.e. a spiro arrangement, at a geminal pair of carbons, and like arrangements. For example, one or two ends of the linker, or the drug or the folate may be connected to the sugar to be inserted into the backbone in a 1,1; 1,2; 1,3; 1,4; 2,3, or other arrangement.

In another embodiment, the hydrophilic linkers, or portions thereof, described herein include primarily carbon, hydrogen, and nitrogen, and have a carbon/nitrogen ratio of about 3:1 or less, or of about 2:1 or less. In one aspect, the hydrophilic linkers described herein include a plurality of amino functional groups.

In another embodiment, the linkers, or portions thereof, include one or more amino groups of the following formulae:

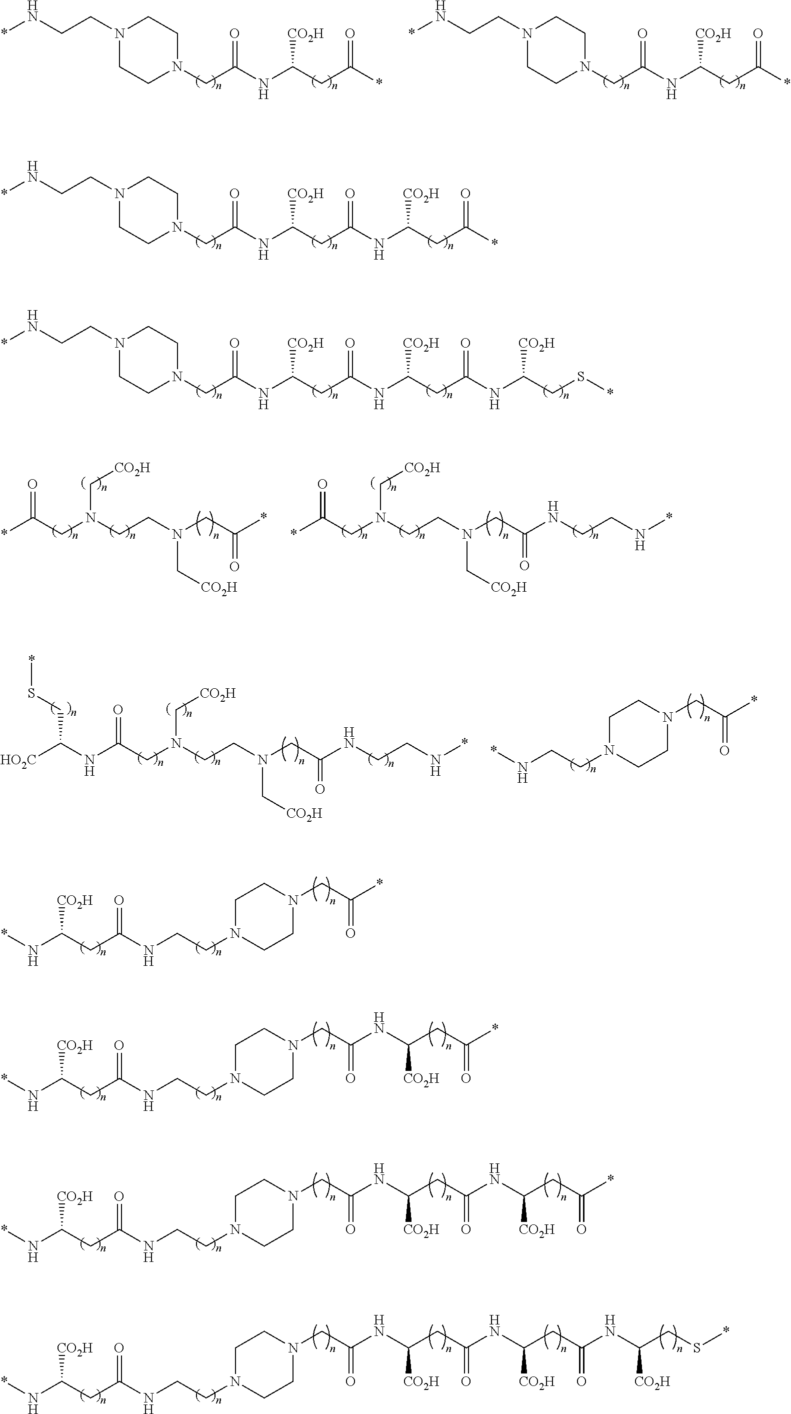

where n is an integer independently selected in each instance from 1 to about 3. In one aspect, the integer n is independently 1 or 2 in each instance. In another aspect, the integer n is 1 in each instance.

In another embodiment, the hydrophilic linker, or a portion thereof, is a sulfuric acid ester, such as an alkyl ester of sulfuric acid. Illustratively, the linker, or a portion thereof, is of the following formula:

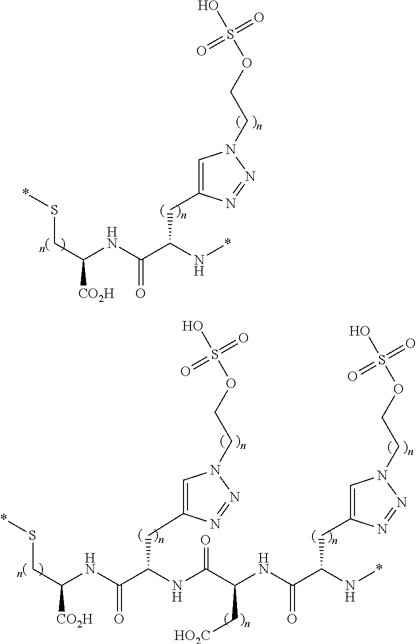

where n is an integer independently selected in each instance from 1 to about 3. Illustratively, n is independently 1 or 2 in each instance.

It is understood, that in such polyhydroxyl, polyamino, carboxylic acid, sulfuric acid, and like linkers that include free hydrogens bound to heteroatoms, one or more of those free hydrogen atoms may be protected with the appropriate hydroxyl, amino, or acid protecting group, respectively, or alternatively may be blocked as the corresponding pro-drugs, the latter of which are selected for the particular use, such as pro-drugs that release the parent drug under general or specific physiological conditions.

In each of the foregoing illustrative examples of linkers, there are also included in some cases additional portions L_{S}, and/or additional releasable linker portions L_{R}. Those linker portions also may include asymmetric carbon atoms. It is to be further understood that the stereochemical configurations shown herein are merely illustrative, and other stereochemical configurations are described. For example in one variation, the corresponding unnatural amino acid configurations may be included in the compound described herein as follows:

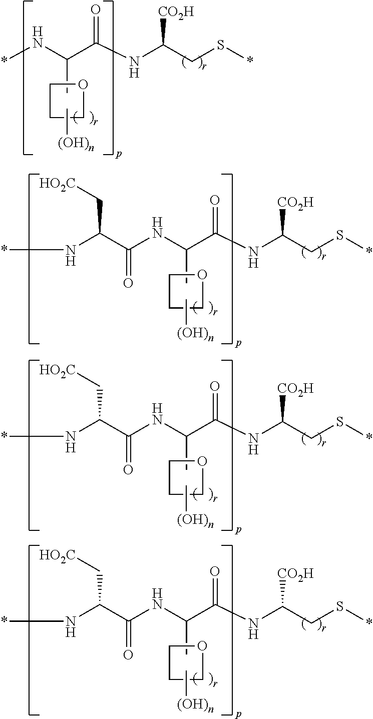

wherein n is an integer from 2 to about 5, p is an integer from 1 to about 5, and r is an integer from 1 to about 4, as described above.

It is to be further understood that in the foregoing embodiments, open positions, such as (*) atoms are locations for attachment of the folate or the drug to be delivered. In addition, it is to be understood that such attachment of either or both of the folate and the drug may be direct or through an intervening linker portions. Intervening linker portions include other linker portions or releasable linker portions. Illustrative additional linker portions and releasable linker portions formed therefrom that can be included in the compounds described herein are described in U.S. Pat. No. 7,601,332, and in U.S. Published Application No. 2010/0323973, the disclosures of which are incorporated herein by reference.

In one embodiment, the hydrophilic linker, or a portion thereof, comprises one or more carbohydrate containing or polyhydroxyl groups. In another embodiment, the hydrophilic linker, or a portion thereof, comprises at least three carbohydrate containing or polyhydroxyl groups. In another embodiment, the hydrophilic linker, or a portion thereof, comprises one or more carbohydrate containing or polyhydroxyl group containing portions, and one or more aspartic acids. In another embodiment, the hydrophilic linker, or a portion thereof, comprises one or more carbohydrate containing or polyhydroxyl group containing portions, and one or more glutamic acids. In another embodiment, the hydrophilic linker, or a portion thereof, comprises one or more carbohydrate containing or polyhydroxyl group containing portions, one or more glutamic acids, one or more aspartic acids, and one or more beta amino alanines. In a series of variations, in each of the foregoing embodiments, the hydrophilic linker, or a portion thereof, also includes one or more cysteines. In another series of variations, in each of the foregoing embodiments, the hydrophilic linker, or a portion thereof, also includes at least one arginine.

In another embodiment, the hydrophilic linker, or a portion thereof, comprises one or more divalent 1,4-piperazines that are included in the chain of atoms connecting the folate with the drug. In one variation, the hydrophilic linker, or a portion thereof, includes one or more carbohydrate containing or polyhydroxyl group containing portions. In another variation, the hydrophilic linker, or a portion thereof, includes one or more carbohydrate containing or polyhydroxyl group containing poritons and one or more aspartic acids. In another variation, the hydrophilic linker, or a portion thereof, includes one or more carbohydrate containing or polyhydroxyl group containing portions and one or more glutamic acids. In a series of variations, in each of the foregoing embodiments, the hydrophilic linker, or a portion thereof, also includes one or more cysteines. In another series of variations, in each of the foregoing embodiments, the hydrophilic linker, or a portion thereof, also includes at least one arginine.

In another embodiment, the hydrophilic linker, or a portion thereof, comprises one or more oligoamide hydrophilic portions, such as aminoethylpiperazinylacetamide.

In another embodiment, the hydrophilic linker, or a portion thereof, comprises one or more triazole linked carbohydrate containing or polyhydroxyl group containing portions. In another embodiment, the hydrophilic linker, or a portion thereof, comprises one or more amide linked carbohydrate containing or polyhydroxyl group containing portions. In another embodiment, the hydrophilic linker, or a portion thereof, comprises one or more PEG groups. In another embodiment, the hydrophilic linker, or a portion thereof, comprises one or more cysteines. In another embodiment, the hydrophilic linker, or a portion thereof, comprises one or more EDTA residues or EDTA derivatives.

In any of the embodiments described herein portions of the linker can be —NR¹—, oxygen, sulfur, and the formulae —(NR¹NR²)—, —SO—, —(SO₂)—, and —N(R³)O—, wherein R¹, R², and R³ are each independently selected from hydrogen, alkyl, aryl, arylalkyl, substituted aryl, substituted arylalkyl, heteroaryl, substituted heteroaryl, and alkoxyalkyl.

Illustrative linkers described herein that are releasable include linkers that include hemiacetals and sulfur variations thereof, acetals and sulfur variations thereof, hemiaminals, aminals, and the like, and can be formed from methylene fragments substituted with at least one heteroatom, 1-alkoxyalkylene, 1-alkoxycycloalkylene, 1-alkoxyalkylenecarbonyl, 1-alkoxycycloalkylenecarbonyl, and the like. Illustrative linkers that are releasable described herein include polyvalent linkers that include carbonylarylcarbonyl, carbonyl(carboxyaryl)carbonyl, carbonyl(biscarboxyaryl)carbonyl, haloalkylenecarbonyl, and the like. Illustrative linkers that are releasable described herein include linkers that include alkylene(dialkylsilyl), alkylene(alkylarylsilyl), alkylene(diarylsilyl), (dialkylsilyl)aryl, (alkylarylsilyl)aryl, (diarylsilyl)aryl, and the like. Illustrative linkers that are releasable described herein include oxycarbonyloxy, oxycarbonyloxyalkyl, sulfonyloxy, oxysulfonylalkyl, and the like. Illustrative linkers that are releasable described herein include linkers that include iminoalkylidenyl, carbonylalkylideniminyl, iminocycloalkylidenyl, carbonylcycloalkylideniminyl, and the like. Illustrative linkers that are releasable described herein include linkers that include alkylenethio, alkylenearylthio, and carbonylalkylthio, and the like. Each of the foregoing fragments is optionally substituted with a substituent X², as defined herein.

The substituents X² can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment a portion of the linker can be nitrogen, and the substituent X² and the nitrogen portion can be taken together with the releasable linker to which they are bound to form an heterocycle.

The heterocycles can be pyrrolidines, piperidines, oxazolidines, isoxazolidines, thiazolidines, isothiazolidines, pyrrolidinones, piperidinones, oxazolidinones, isoxazolidinones, thiazolidinones, isothiazolidinones, and succinimides.

In any of the embodiments described herein, the linker that is releasable may include oxygen bonded to methylene, 1-alkoxyalkylene, 1-alkoxycycloalkylene, 1-alkoxyalkylenecarbonyl, and 1-alkoxycycloalkylenecarbonyl to form an acetal or ketal, wherein each of the fragments is optionally substituted with a substituent X², as defined herein. Alternatively, the methylene or alkylene is substituted with an optionally-substituted aryl.

In any of the embodiments described herein, the linker that is releasable may include oxygen bonded to sulfonylalkyl to form an alkylsulfonate.

In any of the embodiments described herein, the linker that is releasable may include nitrogen bonded to iminoalkylidenyl, carbonylalkylideniminyl, iminocycloalkylidenyl, and carbonylcycloalkylideniminyl to form an hydrazone, each of which is optionally substituted with a substituent X², as defined herein. In an alternate configuration, the hydrazone may be acylated with a carboxylic acid derivative, an orthoformate derivative, or a carbamoyl derivative to form releasable linkers containing various acylhydrazones.

In any of the embodiments described herein, the linker that is releasable may include oxygen bonded to alkylene(dialkylsilyl), alkylene(alkylarylsilyl), alkylene(diarylsilyl), (dialkylsilyl)aryl, (alkylarylsilyl)aryl, and (diarylsilyl)aryl to form a silanol, each of which is optionally substituted with a substituent X², as defined herein.

In any of the embodiments described herein, the linker that is releasable may include nitrogen bonded to carbonylarylcarbonyl, carbonyl(carboxyaryl)carbonyl, carbonyl(biscarboxyaryl)carbonyl to form an amide, or alternatively an amide with a drug nitrogen.

In any of the embodiments described herein, the linker that is releasable may include oxygen bonded to carbonylarylcarbonyl, carbonyl(carboxyaryl)carbonyl, carbonyl(biscarboxyaryl)carbonyl to form an ester, or alternatively an ester with a drug oxygen.

Illustrative linkers that are releasable also include dipeptides of a predetermined sequence that are a substrate for predetermined intracellular enzymes or peptidases. For example, the linker may comprise valanyl-X—, such as Val-Ala-, Val-Lys-, Val-Arg-, and the like, each forming an amide bond in the linker. Without being bound by theory it is believed herein that the Val-X forms at least a part of the sequence necessary to be a substrate of the enzyme or peptidase, such as Cathepsin B, which is capable of cleaving the amide bond formed in the linker. Illustratively, the a linker that is releasable comprises Val-X—NH-Ph-CH₂—O.

The following illustrative bivalent radicals may form part of the linker.

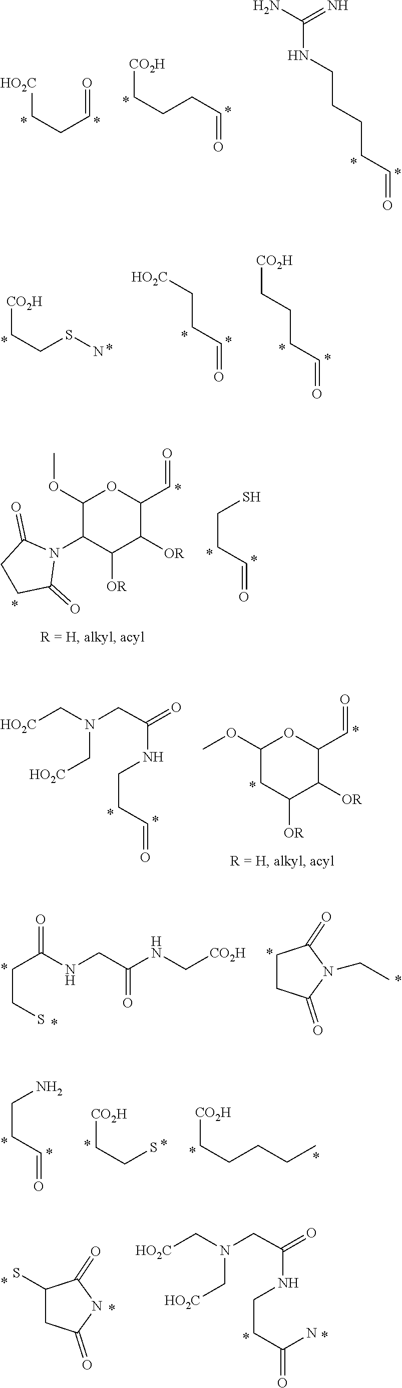

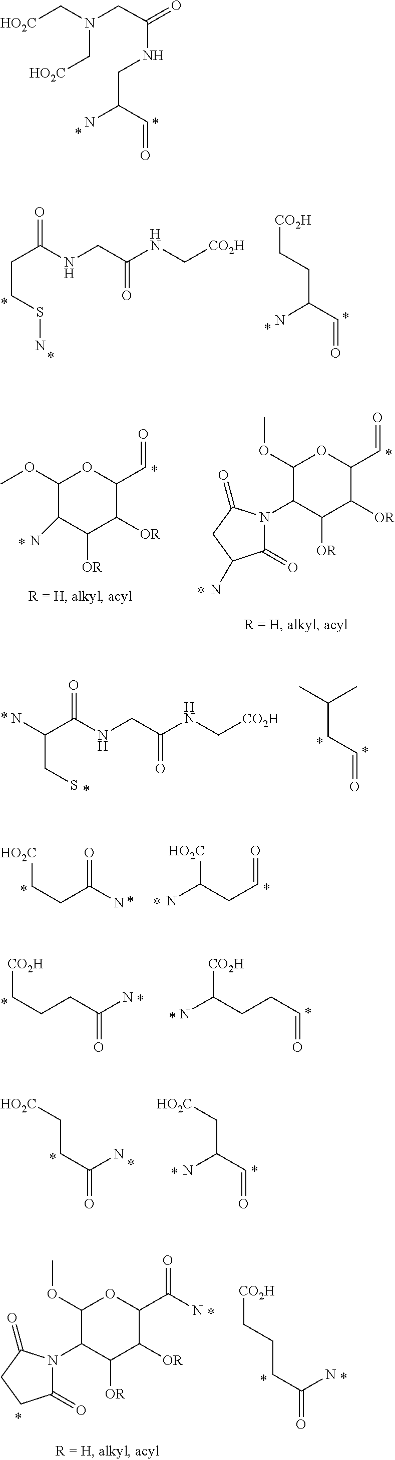

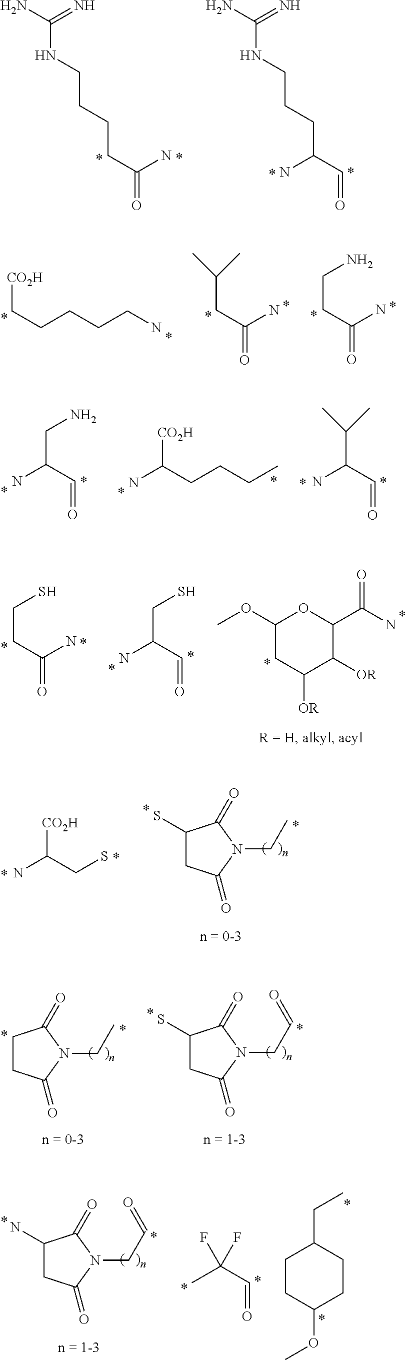

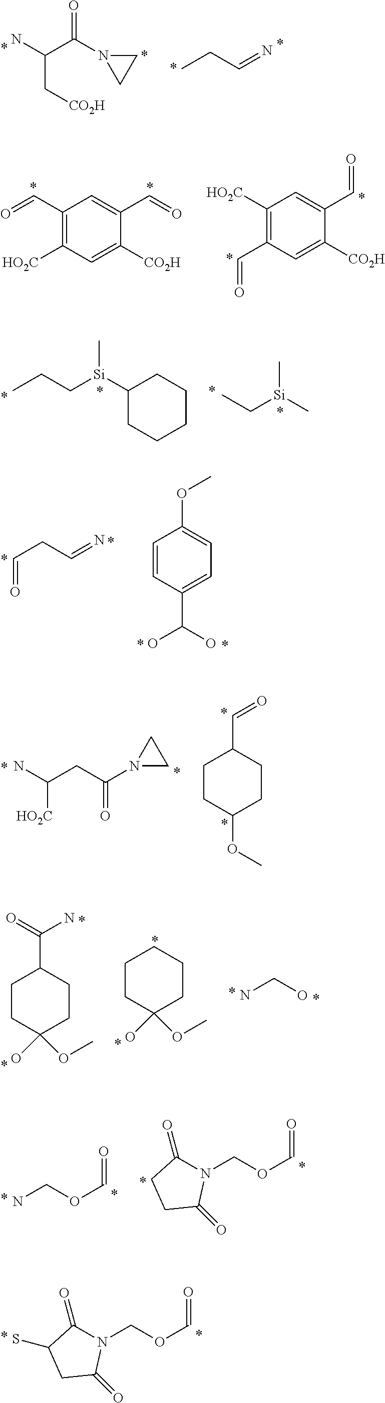

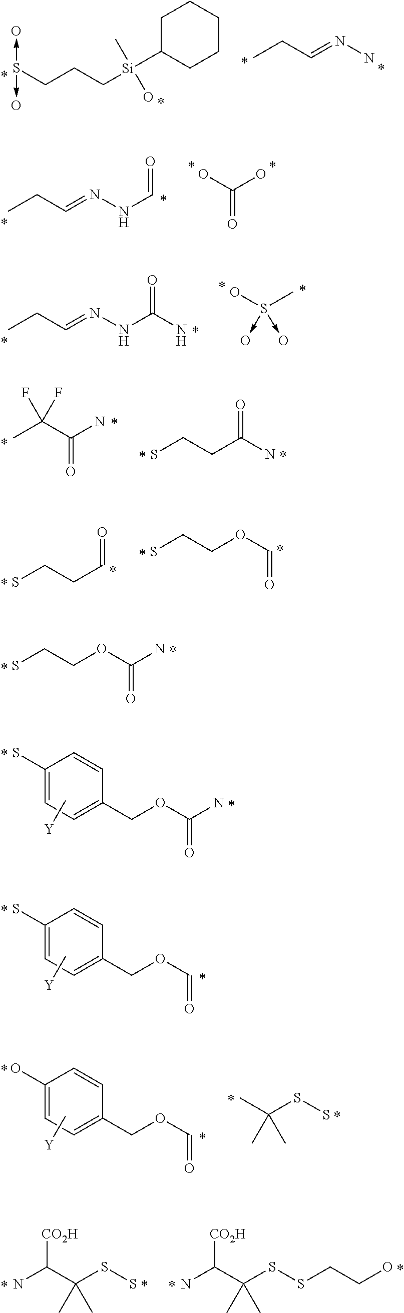

It is to be understood that the linker portion described herein may be combined in any chemically relevant way, either directly or via an intervening heteroatom to construct the linkers that are releasable described herein. It is further understood that the nature of the arrangement of the linker portions defines where the releasable linker will be cleaved in vivo. For example, two linker portions that terminate in a sulfur atom when combined form a disulfide, which is the cleavable bond in the releasable linker formed thereby.

In one aspect, the linker comprises a 3-thiosuccinimid-1-ylalkyloxymethyloxy moiety, where the methyl is optionally substituted with alkyl or substituted aryl.

In another aspect, the linker comprises a 3-thiosuccinimid-1-ylalkylcarbonyl, where the carbonyl forms an acylaziridine with the drug, or analog or derivative thereof.

In another aspect, the linker comprises a 1-alkoxycycloalkylenoxy moiety.

In another aspect, the linker comprises an alkyleneaminocarbonyl(dicarboxylarylene)carboxylate.

In another aspect, the linker comprises a dithioalkylcarbonylhydrazide, where the hydrazide forms an hydrazone with the drug, or analog or derivative thereof.

In another aspect, the linker comprises a 3-thiosuccinimid-1-ylalkylcarbonylhydrazide, where the hydrazide forms a hydrazone with the drug, or analog or derivative thereof.

In another aspect, the linker comprises a 3-thioalkylsulfonylalkyl(disubstituted silyl)oxy, where the disubstituted silyl is substituted with alkyl or optionally substituted aryl.

In another aspect, the linker comprises a plurality of portions selected from the group consisting of the naturally occurring amino acids and stereoisomers thereof.

In another aspect, the linker comprises a 2-dithioalkyloxycarbonyl, where the carbonyl forms a carbonate with the drug, or analog or derivative thereof.

In another aspect, the linker comprises a 2-dithioarylalkyloxycarbonyl, where the carbonyl forms a carbonate with the drug, or analog or derivative thereof, and the aryl is optionally substituted.

In another aspect, the linker comprises a 4-dithioarylalkyloxycarbonyl, where the carbonyl forms a carbonate with the drug, or analog or derivative thereof, and the aryl is optionally substituted.

In another aspect, the linker comprises a 3-thiosuccinimid-1-ylalkyloxyalkyloxyalkylidene, where the alkylidene forms an hydrazone with the drug, or analog or derivative thereof, each alkyl is independently selected, and the oxyalkyloxy is optionally substituted with alkyl or optionally substituted aryl.

In another aspect, the linker comprises a 2-dithioalkyloxycarbonylhydrazide.

In another aspect, the linker comprises a 2- or 3-dithioalkylamino, where the amino forms a vinylogous amide with the drug, or analog or derivative thereof.

In another aspect, the linker comprises a 2-dithioalkylamino, where the amino forms a vinylogous amide with the drug, or analog or derivative thereof, and the alkyl is ethyl.

In another aspect, the linker comprises a 2- or 3-dithioalkylaminocarbonyl, where the carbonyl forms a carbamate with the drug, or analog or derivative thereof.

In another aspect, the linker comprises a 2-dithioalkylaminocarbonyl, where the carbonyl forms a carbamate with the drug, or analog or derivative thereof. In another aspect, the alkyl is ethyl.

In another aspect, the linker comprises a 2-dithioalkyloxycarbonyl, where the carbonyl forms a carbamate with the drug, or analog or derivative thereof. In another aspect, the alkyl is ethyl.

In another aspect, the linker comprises a 2-dithioarylalkyloxycarbonyl, where the carbonyl forms a carbamate or a carbamoylaziridine with the drug, or analog or derivative thereof.

In another aspect, the linker comprises a 4-dithioarylalkyloxycarbonyl, where the carbonyl forms a carbamate or a carbamoylaziridine with the drug, or analog or derivative thereof.

In another embodiment, the linkers described herein comprise portions of the following formulae

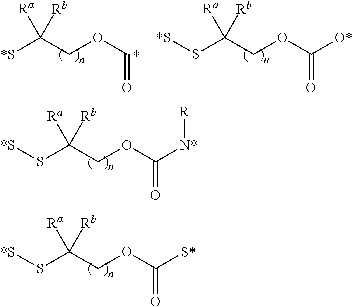

where n is an integer selected from 1 to about 4; Ra and Rb are each independently selected from the group consisting of hydrogen and alkyl, including lower alkyl such as C₁-C₄ alkyl that are optionally branched; or Ra and Rb are taken together with the attached carbon atom to form a carbocyclic ring; R is an optionally substituted alkyl group, an optionally substituted acyl group, or a suitably selected nitrogen protecting group; and (*) indicates points of attachment for the drug, other linker portions, or other parts of the compound.

In another embodiment, the linkers described herein comprise portions of the following formulae

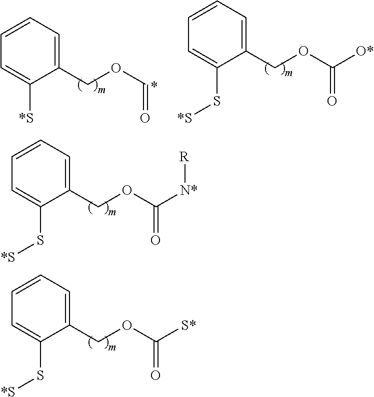

where m is an integer selected from 1 to about 4; R is an optionally substituted alkyl group, an optionally substituted acyl group, or a suitably selected nitrogen protecting group; and (*) indicates points of attachment to the drug, other linker portions, or other parts of the compound.

In another embodiment, the linkers described herein comprise portions of formulae

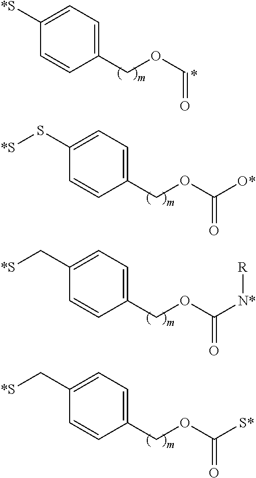

where m is an integer selected from 1 to about 4; R is an optionally substituted alkyl group, an optionally substituted acyl group, or a suitably selected nitrogen protecting group; and (*) indicates points of attachment to the drug, other linker portions, or other parts of the compound.

In another embodiment, the compounds described herein comprise one or more linkers of selected from the formulae:

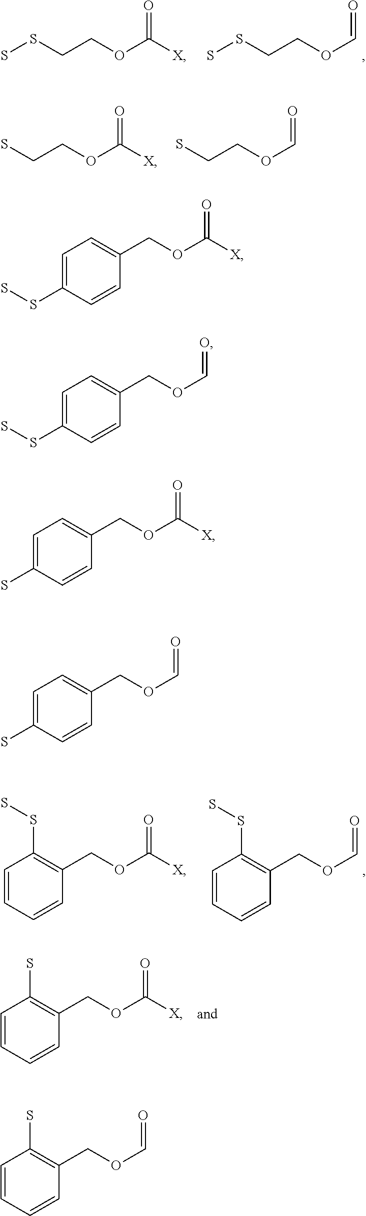

wherein X is NH, O, or S.

In another embodiment, the linkers herein described comprise a radical having the formula:

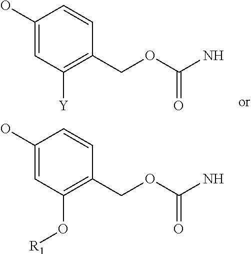

In another embodiment, the linkers described herein comprise a radical having the formula:

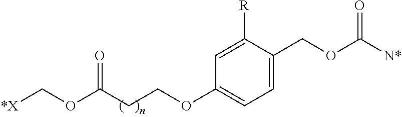

where X is an heteroatom, such as nitrogen, oxygen, or sulfur, n is an integer selected from 0, 1, 2, and 3, R is hydrogen, or a substituent, including a substituent capable of stabilizing a positive charge inductively or by resonance on the aryl ring, such as alkoxy, and the like, and the symbol (*) indicates points of attachment. It is appreciated that other substituents may be present on the aryl ring, the benzyl carbon, the alkanoic acid, or the methylene bridge, including but not limited to hydroxy, alkyl, alkoxy, alkylthio, halo, and the like.

In another embodiment, the linkers, or portions therof, described herein comprise a group selected from the group consisting of carbonyl, thionocarbonyl, alkylene, cycloalkylene, alkylenecycloalkyl, alkylenecarbonyl, cycloalkylenecarbonyl, carbonylalkylcarbonyl, 1 alkylenesuccinimid-3-yl, 1 (carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each of said groups is optionally substituted with one or more substituents X1; wherein each substituent X¹ is independently selected from the group consisting of alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from the group consisting of an amino acid, an amino acid derivative, and a peptide, and wherein R⁶ and R⁷ are each independently selected from the group consisting of an amino acid, an amino acid derivative, and a peptide.

In another embodiment, the compounds described herein comprise one or more unnatural amino acids.

In another embodiment, the compounds described herein comprise one or more unnatural amino acids wherein at least one unnatural amino acid has the D-configuration.

In another embodiment, the compounds described herein comprise at least one unnatural amino acid selected from D-alanine, D-aspartic acid, D-asparagine, D-cysteine, D-glutamic acid, D-phenylalanine, D-histidine, D-isoleucine, D-lysine, D-leucine, D-methionine, D-proline, D-glutamine, D-arginine, D-serine, D-threonine, D-valine, D-tryptophan, D-tyrosine, and D-ornithine, or a derivative thereof.

In another embodiment, the compounds described herein comprise at least one unnatural amino acid selected from D-aspartic acid, D-asparagine, D-cysteine, D-glutamic acid, D-histidine, D-lysine, D-methionine, D-glutamine, D-arginine, D-serine, D-threonine, D-tryptophan, D-tyrosine, and D-ornithine, or a derivative thereof.

In another embodiment, the compounds described herein comprise at least one unnatural amino acid selected from D-aspartic acid, D-asparagine, D-cysteine, D-glutamic acid, D-histidine, D-lysine, D-glutamine, D-arginine, D-serine, D-threonine, D-tryptophan, and D-ornithine, or a derivative thereof.

In another embodiment, the compounds described herein comprise at least one unnatural amino acid selected from D-aspartic acid, D-cysteine, D-glutamic acid, D-lysine, D-arginine, D-serine, and D-ornithine, or a derivative thereof.

In another embodiment, the compounds described herein comprise two or more unnatural amino acids.

In another embodiment, the compounds described herein comprise three or more unnatural amino acids.

In another embodiment, the compounds described herein comprise four or more unnatural amino acids.

In another embodiment, the compounds described herein further comprise one or more disulfides.

In another embodiment, the compounds described herein comprise at least one disulfide comprising D-cysteinyl.

As used herein the term “radical” with reference to, for example, a drug or a folate, refers to a drug or a folate, as described herein, where one or more atoms or groups, such as a hydrogen atom or an alkyl group on a heteroatom, or a hydroxyl group on a carboxylic acid group, and the like, is removed to provide a radical for conjugation to the linker.

As used herein, the term “releasable linker” or “linker that is releasable” refers to a linker that includes at least one bond that can be broken under physiological conditions, such as a pH-labile, acid-labile, base-labile, oxidatively-labile, metabolically-labile, biochemically-labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It is understood that a cleavable bond can connect two adjacent atoms within the releasable linker and/or connect other linker portions or the folate and/or the drug, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, following breakage of the bond, the releasable linker is separated from the other moiety.

Illustrative embodiments of “a folate” or “the folate” or “folates” (terms which are used interchangeably) include folic acid, and analogs and derivatives of folic acid, such as folinic acid, pteroylpolyglutamic acid, pteroyl-D-glutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. Other folates useful as complex forming ligands for this invention are the folate receptor-binding analogs aminopterin, amethopterin (also known as methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). Additional folates (for example, analogs of folic acid) that bind to folate receptors are described in U.S. Patent Application Publication Nos. 2005/0227985 and 2004/0242582, the disclosures of which are incorporated herein by reference. Folic acid, and the foregoing analogs and/or derivatives are also termed “a folate,” “the folate,” or “folates” reflecting their ability to bind to folate-receptors, and such ligands when conjugated with exogenous molecules are effective to enhance transmembrane transport, such as via folate-mediated endocytosis as described herein. The foregoing are included in the folate receptor binding compounds described herein.

It is appreciated that compounds described herein may exist in unsolvated forms as well as solvated forms, including hydrated forms. In general, the solvated forms are equivalent to unsolvated forms and are encompassed within the scope of the present invention.

In another embodiment, compositions and/or dosage forms for administration of the compound are prepared from the compound with purity of at least about 90%, or about 95%, or about 96%, or about 97%, or about 98%, or about 99%, or about 99.5%. In another embodiment, compositions and or dosage forms for administration of the compound are prepared from the compound with a purity of at least 90%, or at least 95%, or at least 96%, or at least 97%, or at least 98%, or at least 99%, or at least 99.5%.

### EXAMPLES

The following abbreviations are used herein: ATC (anaplastic thyroid cancer), FACS (fluorescence-activated cell sorting, flow cytometry), FR (folate receptor), FR(−) (folate receptor negative), FR(+) (folate receptor positive), and TAM (tumor-associated macrophage). Antibodies used for FACS analyses: Rat CD11b (clone WT.5; BD Pharmingen), Rat MHCII (clone OX17; eBioscience), Rat CD163 (clone ED2; AbD Serotec), Mouse CD11b (clone M1/70; eBioscience), mouse F4/80 (clone BM8; eBioscience), mouse MHCII (clone M5/114.15.12; eBioscience), Mouse FRβ (clone 17B6; antibody obtained from Endocyte Inc.). EC0431 (a folate biotin conjugate) and EC0486 (a folate Cy5.5 conjugate) (100 nM final concentration; obtained from Endocyte Inc.) for staining of functional folate receptors, Streptavidin-PECy7 for staining of folate-biotin conjugates, such as EC0431 (eBioscience).

EXAMPLE 1. Optical imaging of 4T1 mammary tumors in nude rats. Female Foxn1^{nu} nude rats (Harlan, Inc., Indianapolis, Ind.) are subcutaneously implanted with 0.95×10⁶ 4T1 tumor cells in the left mammary region. The animals are switched to a folate-deficient diet 4 days later. On day 9 post the initial tumor implantation, animals are intravenously dosed with 100 nmol/kg of a folate-Cy5.5 conjugate (for example, EC0486) without (n=2) and with (n=1) a 400-fold excess of a high-affinity folate competitor (for example, pteroyl-γGlu-D-Asp-D-Asp (EC0923)). Processes for preparing EC0923 are described in WO 2014/062697, the disclosure of which is incorporated herein by reference. Two hours later, tumor, kidney, and muscle are harvested from each animal and imaged using IVIS Lumina imaging system (excitation: 710 nm; emission: ICG). The kidney and muscle from a healthy rat are also used as the negative control.

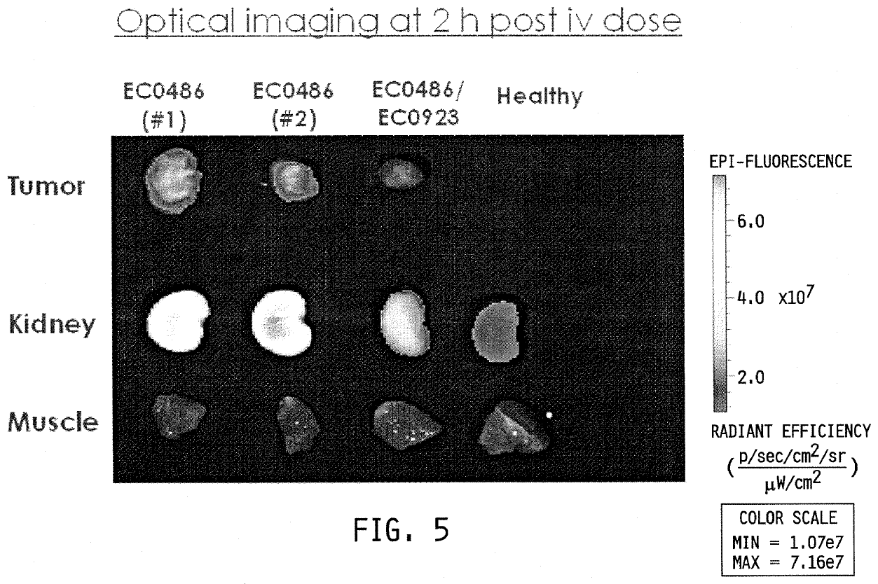

Uptake of the folate-Cy5.5 conjugate in FR(−) 4T1 tumors. It is appreciated herein that the murine 4T1 breast carcinoma is a model for the triple negative breast cancer in humans because the carcinoma is highly aggressive and metastatic. The tumor cells themselves do not express any functional FR. In nude rats bearing 4T1 mammary tumors, intravenously administered EC0486 is taken up by 4T1 tumors and the uptake is specifically blocked by the folate competitor EC0923 (FIG. 5). FR(−) specific uptake is seen in the kidney of the same animals, while no uptake is seen in muscle or tissues from a healthy animal. Those observations suggest that tumor stroma cells such as FR(−) expressing TAMs may have contributed to the tumor retention of the folate imaging agent.

EXAMPLE 2. FACS analysis of 4T1 mammary tumor cells post EC0486 imaging. The 4T1 xenograft tumors harvested 2 h after EC0486 dosing in nude rats (FIG. 5) are subjected to FACS analysis after an enzymatic digestion step to generate single-cell suspensions. The tumor suspension cells are stained for macrophage markers (CD163 and CD11 b) and analyzed on the same day. To compare functional FR levels, KB cells are stained with EC0486 and used as a positive control. In addition, an aliquot of the tumor suspension cells are stained ex-vivo with EC0486 to saturate any unoccupied FRs on TAMs.

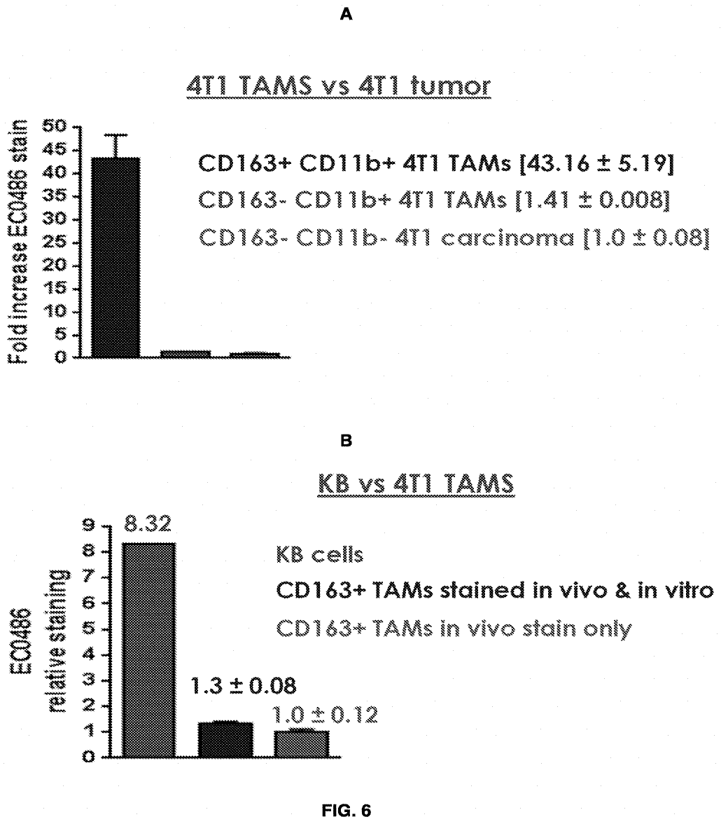

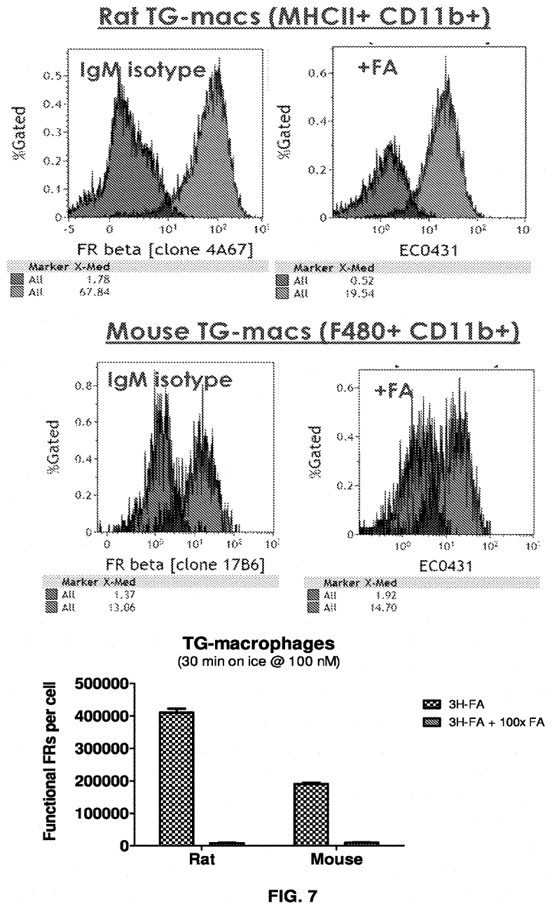

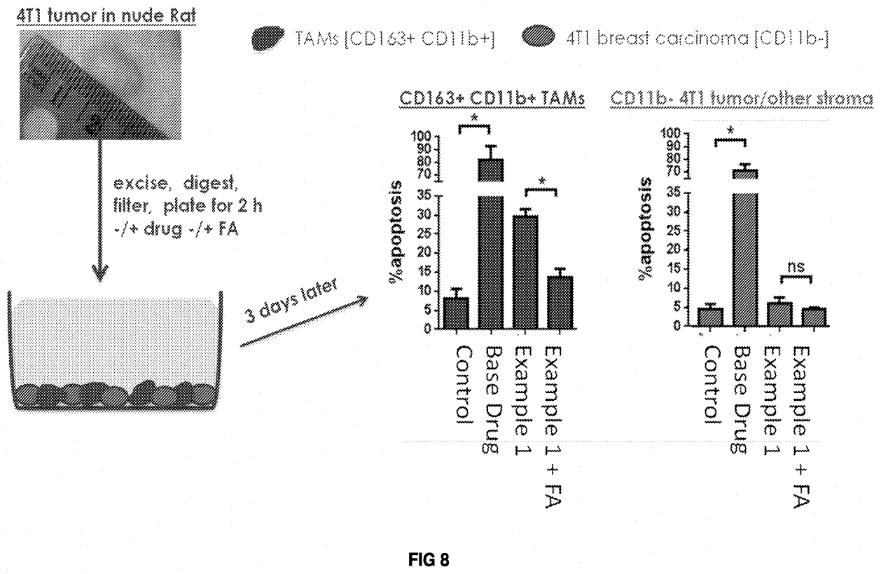

Consistent with FR expression by TAMs, FACS analysis of 4T1 tumors excised from rats dosed intravenously with EC0486 exhibits specific fluorescent staining of the CD163(+) CD11b(+) TAM subpopulation (FIG. 6A). In contrast, negligible levels of cell-associated EC0486 fluorescence is detected in the CD163(−) CD11b(+) 4T1 TAM population and the FR(−) 4T1 tumor cells (FIG. 6A), which confirms folate targeting of fluorophore specifically to the M2 macrophage population. Compared to KB cells that express approximately 4-5 million functional folate receptors per cell, the level of functional FR on CD163(+) CD11b(+) 4T1 TAMs is about 8-fold lower (FIG. 6B). In addition, ex-vivo staining of 4T1 tumor suspension with EC0486 does not increase the fluorescence intensity on 4T1 TAMs, suggesting that all available functional FRs are bound to EC0486 in-vivo (FIG. 6B).

EXAMPLE 3. Activity of Example 1 against 4T1 TAMs ex-vivo. Subcutaneous 4T1 mammary tumors are harvested from nude rats and enzymatically digested to generate single-cell suspensions. On the same day of harvest, whole tumor suspension cells are treated with 100 nM of Example 1, a folic acid-releasable linker-drug conjugate, with and without 100-fold excess of folic acid and the parent drug EC2078. Three days later, the tumor suspension cells are stained for macrophage markers (CD163 and/or CD11b), cell viability (propidium iodide), and late and early apoptosis (Annexin V).

Example 1 showed activity against FR(+) 4T1 TAMs ex-vivo. EC2078, the parent drug of Example 1, induces similar degrees of apoptosis in both TAM and non-TAM cell populations in 4T1 tumor cell suspensions (FIG. 8). However, Example 1 is only effective against the CD163(+)-CD11b(+) 4T1 TAMs (FIG. 8) that are previously shown to express a functional FR (FIG. 6A and FIG. 6B). The observed Example 1 activity is substantially blocked by excess folic acid, suggesting that the effect is at least partially FR-mediated.

EXAMPLE 4. Example 1 activity against 4T1 TAMs in-vivo. As a proof-of-concept study, female Foxn1^{nu} nude rats (Harlan, Inc., Indianapolis, Ind.) on a folate-deficient diet are subcutaneously implanted with 1×10⁶ 4T1 tumor cells in the left and right mammary regions (2 tumors per animal). When the tumors reach about 900-1000 mm³, the animals are intravenously dosed with (i) PBS (n=1), (ii) 100 nmol/kg of the folic acid-releasable linker-drug conjugate, such as Example 1 (n=1), (iii) 100 nmol/kg of Example 1 plus 50 μmol/kg of EC0923 (n=1), or (iv) 50 μmol/kg of EC0923 (n=1). Four days later, the tumors are harvested, enzymatically digested, and subjected to FACS analysis. The tumor cell suspensions are stained for macrophage markers (CD163 and/or CD11b), cell viability (propidium iodide), and late and early apoptosis (Annexin V).

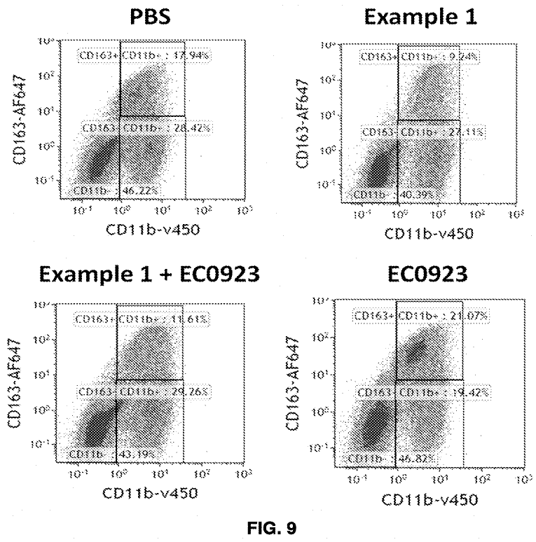

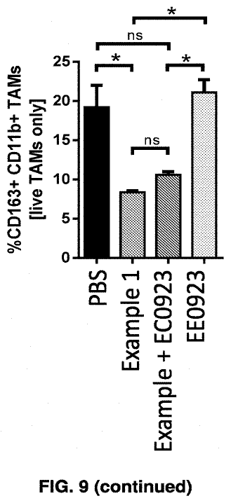

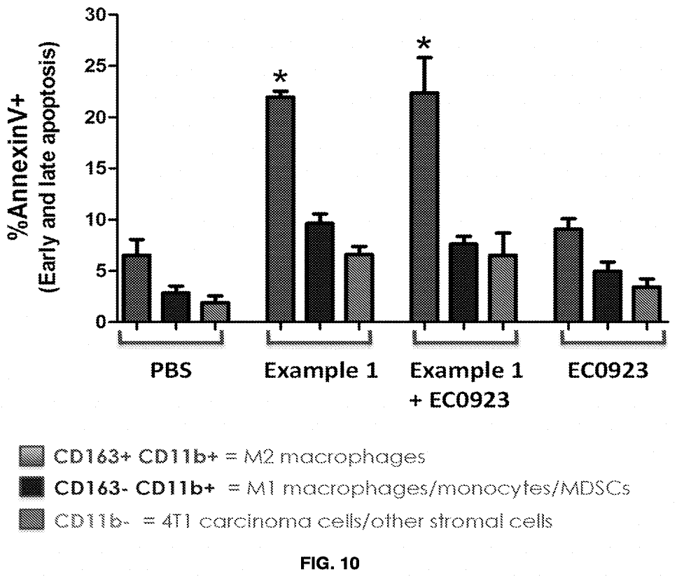

Example 1 demonstrates in-vivo selectivity for FR(+) 4T1 TAMs over FR(−) 4T1 tumor cells. With a single administration, Example 1 shows a significant decrease in the viable CD163(+) CD11b(+) TAM population in 4T1 tumor cells (FIG. 9). While the folate competitor EC0923 alone does not have any effect on 4T1 TAMs, the anti-TAM activity of Example 1 is not blocked by excess EC0923 (FIG. 9). Without being bound by theory, it is believed herein that the lack of competition and FR-specificity of Example 1 in-vivo may be due to early release of the drug from the folic acid-releasable linker-drug conjugate of Example 1. However, Example 1 does not display the same level of in vivo reduction of FR(−) cell populations including CD163(−)-CD11b(+) 4T1 TAMs nor 4T1 tumor cells themselves. In contrast, an increase is observed in apoptotic CD163(+)-CD11b(+) TAMs within 4T1 tumors harvested from rats dosed with Example, but not with either PBS nor EC0923 alone (FIG. 10).

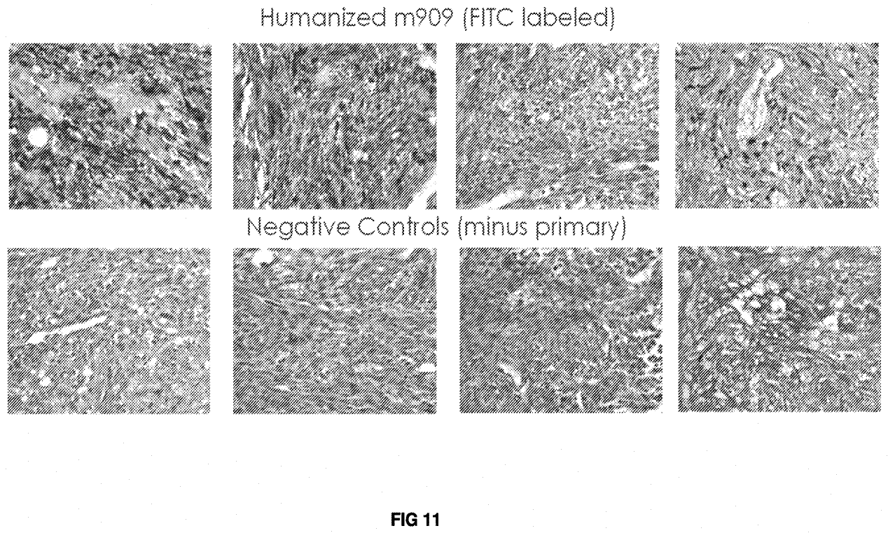

EXAMPLE 5. Immunohistochemical analysis of FR-β expression in ATC. Human anaplastic thyroid cancers (ATC) are highly enriched with CD163(+) and CD68(+) TAMs, comprising up to 70% of the entire tumor mass. This disease is one of the most aggressive forms of solid tumors, and host animals having this disease do not typically survive beyond about 4-6 months. Human ATC tissues are obtained from Advanced Tissue Services (Phoenix, Ariz.). FITC-labeled humanized anti-human FRβ (m909) monoclonal antibodies (obtained from Purdue University, West Lafayette, Ind.) are used to stain ATC tissues after a gentle antigen retrieval step. IHC analysis is performed according to conventional methods. All images are obtained from the same specimen (40×). Preliminary IHC analysis shows a high FR-β protein expression in human anaplastic thyroid cancer specimens (FIG. 11).

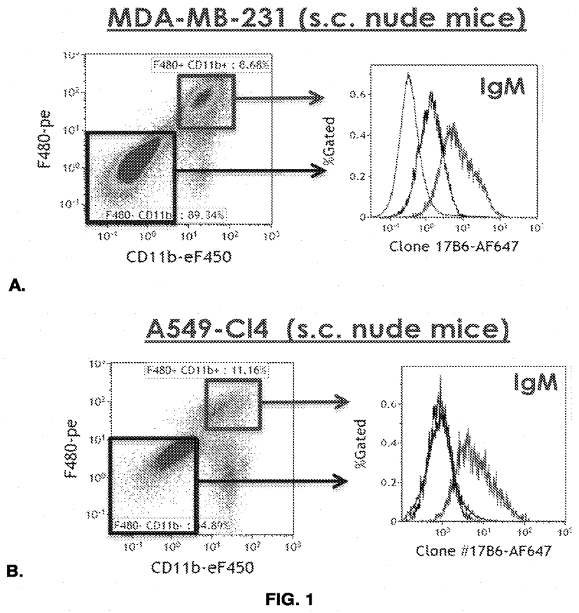

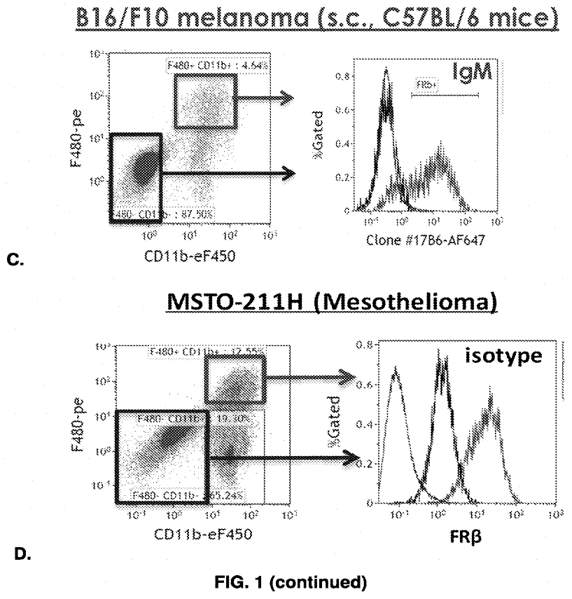

EXAMPLE 6. FRβ expression in human xenograft tumors implanted in nude mice. Human breast cancer (MDA-MB-231 cells), non-small-cell lung cancer (A549 cells), mesothelioma (MSTO-211H cells), melanoma (B16/F10 cells), and Lewis lung carcinoma xenograft tumors implanted in nude mice, and TAMs are identified by FACS using the mouse macrophage markers F4/80(+) and CD11b(+), as shown in FIG. 1. In all three mouse models of human xenograft tumors, only the F4/80(+) CD11b(+) TAMs express FRβ protein. It is appreciated that staining using recently generated anti-mouse FRβ antibodies is specific when compared to an isotype control. Additionally no other cells within the tumors express FRβ (neither F4/80(−) CD11b(−) nor F4/80(−) CD11b(+) cells).

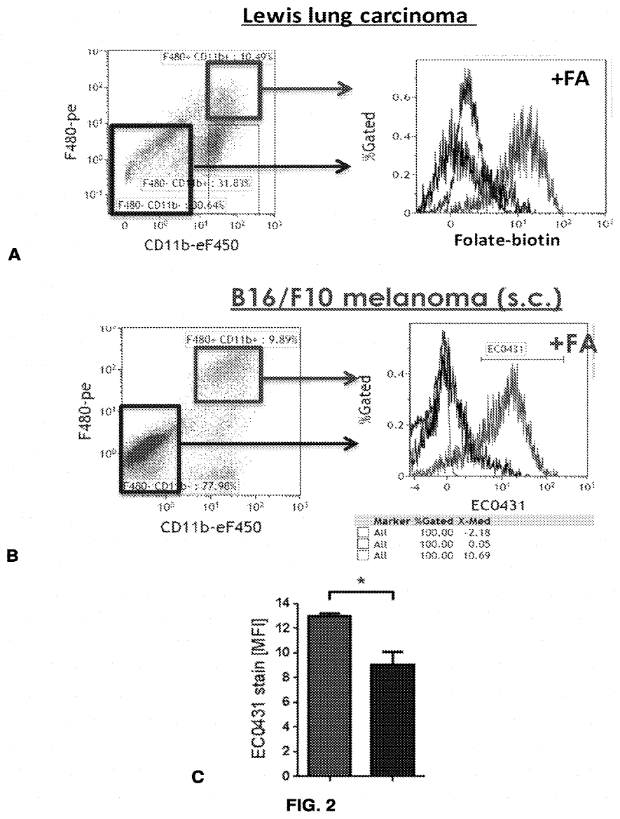

EXAMPLE 7. Functional FRs are detected on both M1 and M2 F4/80(+)-CD11b(+) TAMs in syngeneic mouse models. Mouse TAMs (F4/80(+)-CD11b(+) cells) are identified by FACS in syngeneic Lewis lung carcinoma (LLC) and melanoma (B16/F10) mouse models (FIG. 2). In both models, the TAMs possess a functional folate receptor which is receptive to staining with EC0431 detected with a commercially available streptavidin-linked fluorophore (PE-Cy7). FIG. 2 shows that EC0431 stained the F4/80(+)-CD11b(+) macrophages in a manner which is fully competable in the presence of excess folic acid (xsFA). The data demonstrate that all other cells within the tumor which are not macrophages (identified in the dot plot as either F4/80(−) CD11b(−) or F4/80(−) CD11b(+) cells) are absent for functional folate receptor. FIG. 2C shows a histogram where both M1 (left bar) and M2 (right bar) subsets of TAMs express functional FRs, indicating that folate-targeted compounds may be targeted to both M1 and M2 mouse TAMs.

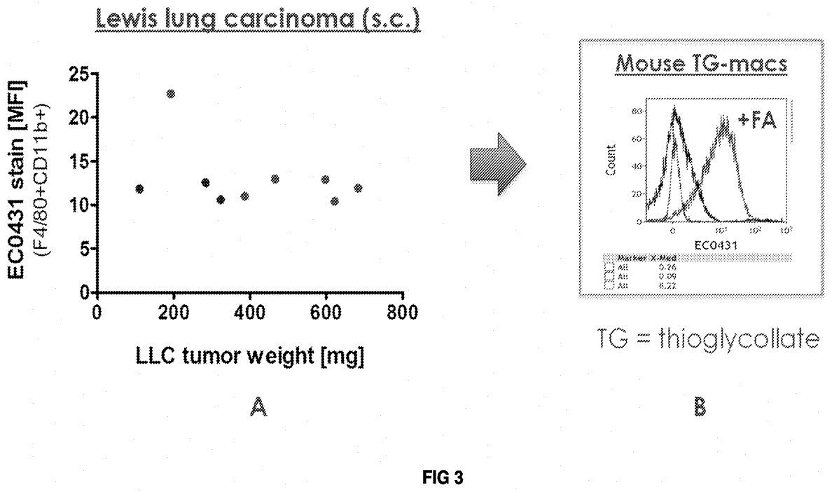

EXAMPLE 8. Functional FR levels on TAMs do not appear to correlate with tumor weights. In the LLC tumor model (FIG. 2A) described in Example 7, levels of functional FR as seen by EC0431 staining are determined and levels of FR on the TAMs are shown to be consistently the same, regardless of the size of the tumors (FIG. 3). This suggests that treatment with a folate linked small molecule should be able to target TAMs in tumors of all sizes, small and large. The histogram overlays in FIG. 3B demonstrate that the levels of functional FR found on LLC TAMs is similar to, if not slightly greater than those found in peritoneal macrophages isolated from the mouse thioglycollate induced peritonitis model of inflammation (compare EC0431 stain around 10 for TAMs in line graph in FIG. 3A to the mouse thioglycollate induced macrophages stained at approximately 8.22 seen in the right-hand trace in FIG. 3B).

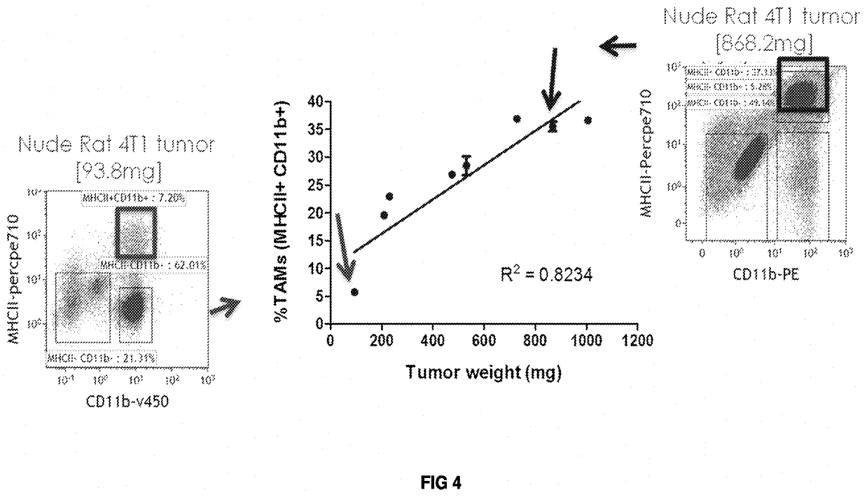

EXAMPLE 9. TAM density in nude rats bearing 4T1 mammary carcinoma correlates with tumor weight (≤1000 mg). FIG. 4 shows data from a nude rat model of solid murine breast tumor line, 4T1. Isolation of single cell suspensions from these rat tumors showed the identification of TAMs as MHCII(+) CD11b(+) cells (FIG. 4; dot plots). The number of TAMs increased as the tumor increased in size (FIG. 4; line graph).

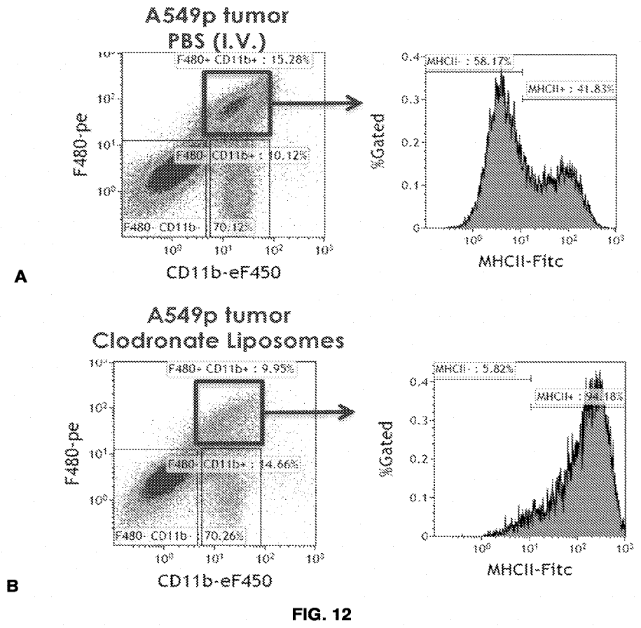

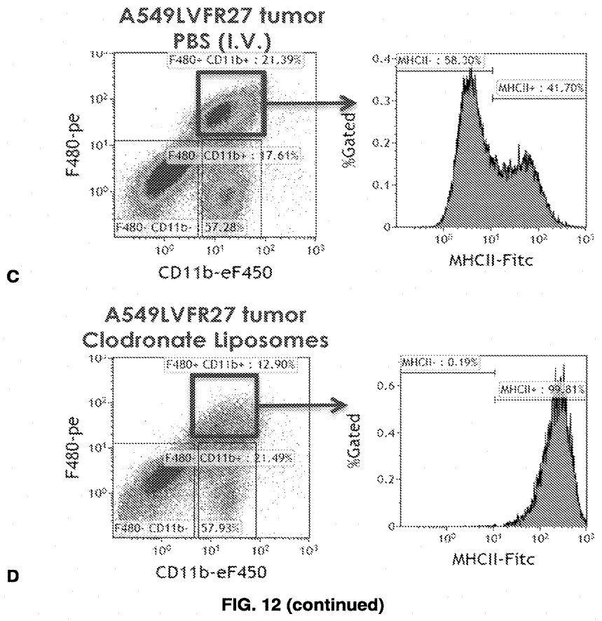

EXAMPLE 10. M2 macrophages are specifically depleted by clodronate liposomes. Two human lung cancer lines (A549, A549LVFR) implanted into nude mice are used to generate the data in FIG. 12. Both lines are variants of the human non-small cell lung cancer (NSCLC). The resulting tumors generated in these mice are harvested and single cell preparations are generated. The total TAMs are identified (F4/80(+) CD11b(+)) using FACS analysis (FIG. 12; left top and left bottom dot plots). The F4/80(+) CD11b(+) TAMs are further determined to contain two subsets of macrophages: 1) the M1 macrophages are the MHCII(+) subset and 2) M2 macrophages are the MHCII-subset (Wang et al. BMC Immunology 2011, 12:43). After intravenous treatment of clodronate liposomes to deplete macrophages from the tumors (FIGS. 12B and 12D, dot plots), the M2 subset of macrophages (MHCII-) are specifically depleted by the clodronate liposomes whereas the M1 subset of macrophages seemed to be unchanged by this treatment.

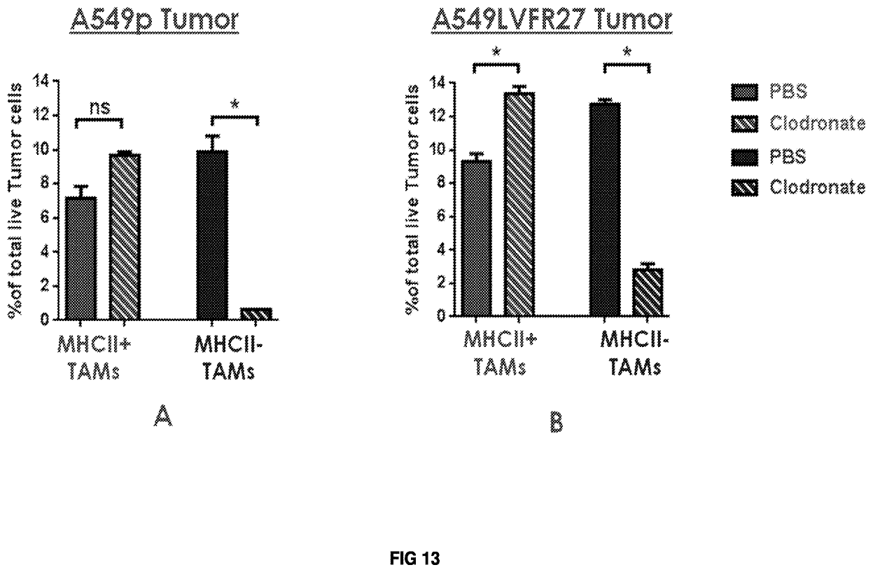

EXAMPLE 11. M2 macrophages are specifically depleted by clodronate liposomes. In FIG. 13, bar graphs show that the number of M2 macrophages from the untreated A549p and A549LVFR27 xenograft tumors (solid bars, right group in each graph; MHCII-TAMs) decreased after treatment with clodronate liposomes (hatched bars; right group in each graph; MHCII-TAMs). It is also shown that M1 macrophages from the untreated xenograft tumors (solid bars, left group in each graph; MHCII(+) TAMs) do not change significantly after treatment with clodronate liposomes (hatched bars, left group in each graph; MHCII(+) TAMs).

EXAMPLE 12. Preparation of B16/F10 and LLC tumors isolated from syngeneic mice. The semi-solid LLC tumors are excised, weighed and crushed through a 40 μm nylon filter in cold PBS to prepare a single cell suspension. Cells are pelleted, discarded supernatant, and resuspended in RBC lysis 5 min at room temperature to lyse RBCs. Cells are washed with cold PBS, pelleted, and resuspended in cold PBS. Then, cells are re-filtered with a 40 μm nylon filter to remove clumps. Pelleted cells are then blocked in FACS stain for >20 min on ice in dark −/+20 μM folate. Pelleted cells are then stained in 100 μL FACS stain containing antibodies for 20 min on ice in the dark. Washed samples are pelleted and either stained with secondary antibodies for 20 min on ice or resuspended in 200 μL PBS+3 μM propidium iodide for immediate FACS analysis. Cells stained with secondary antibodies are washed, pelleted and resuspended in 200 μL PBS+3 μM propidium iodide for immediate FACS analysis.

EXAMPLE 13. Preparation of 4T1 xenograft tumors from nude rats and MDA-MB-231 tumors isolated from nude mice and A549 tumors isolated from nude mice. Each tumor is minced and placed into 20 mL of RPMI1640 (serum free) containing 0.5 mg/mL Collagenase IV, 0.05 mg/mL hyaluronidase, and 0.1 mg/mL DNaseI and placed in a shaker at 200 rpm for 1 hr at 37° C. After 1 hr, the cells/debris/undigested tumor is pelleted at 400×g for 5 min, resuspended in 10 mL RBC lysis solution for 5 min at room temperature, washed with 40 mL cold PBS, pelleted, resuspended in 40 mL PBS, filtered through 40 μm nylon, pelleted, and blocked in FACS stain for >20 min on ice. The pelleted cells are then stained in 100 μL FACS stain containing antibodies for 20 min on ice in the dark, washed, and resuspended in 200 μL PBS+3 μM propidium iodide for immediate FACS analysis.

EXAMPLE 14. Generation of mouse thioglycollate induced peritoneal inflammatory macrophages. Three days after Balb/c mice are injected intraperitoneally with 50 mL/kg with 7.5% thioglycolate medium supplemented with 12.5 mg/mL AGE-BSA, the peritoneal cells are harvested with PBS containing 5 mM EDTA. The cells are pelleted, the RBCs are lysed for 5 min at room temperature, and the cells are washed, pelleted, and resuspended in cold PBS, filtered through 40 μm nylon, and then the cells are counted. FACS stain is performed with respective antibodies to identify the macrophages in addition to 100 nM EC0431 according to a conventional FACS method.

### Compound Example 1

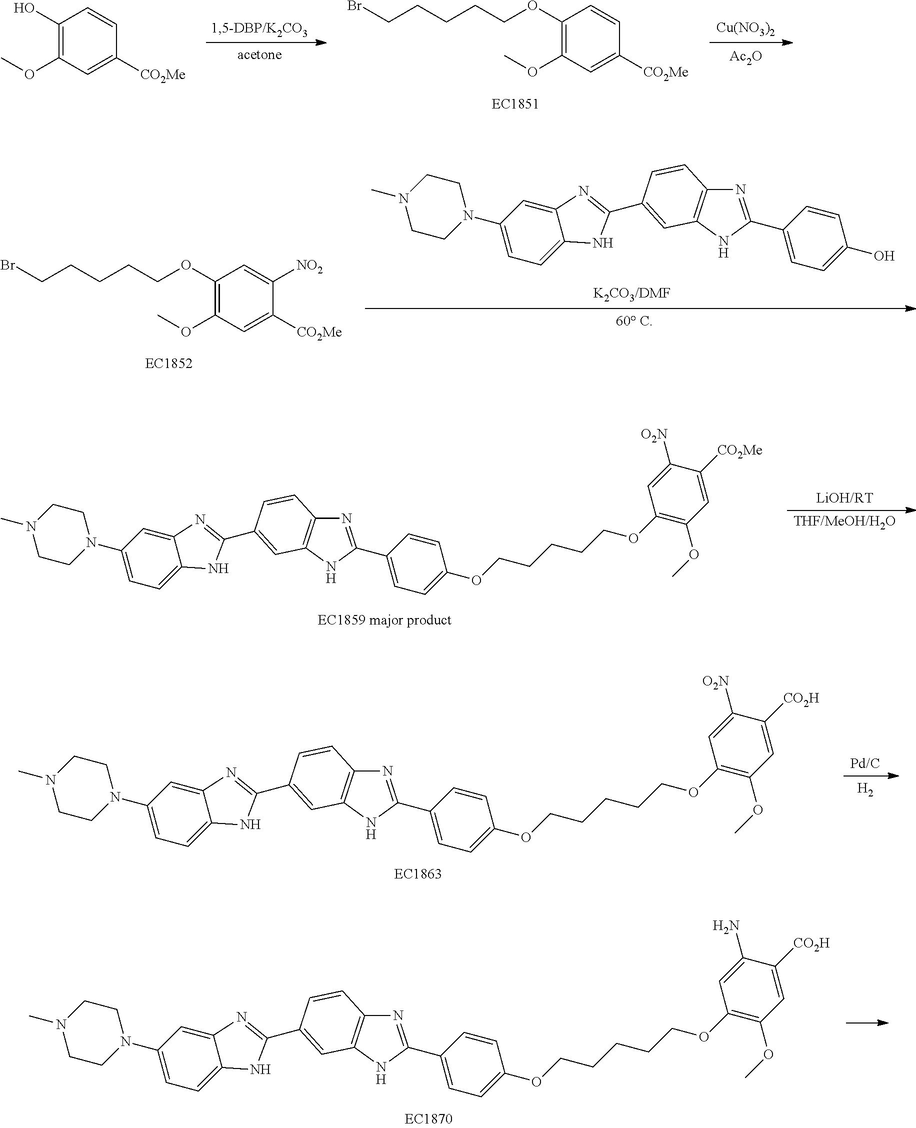

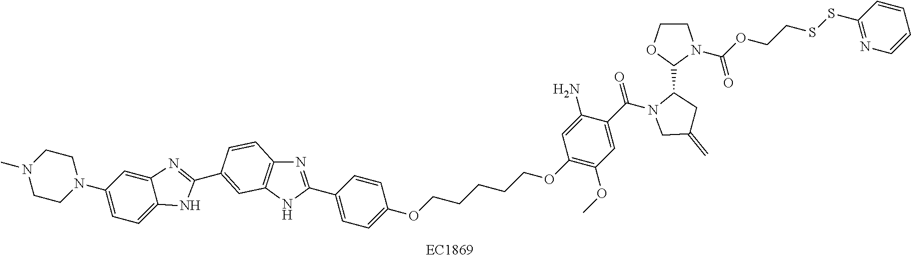 [SMILES: C=C1C[C@@H](C2OCCN2C(=O)OCCSSc2ccccn2)N(C(=O)c2cc(OC)c(OCCCCCOc3ccc(-c4nc5ccc(-c6nc7cc(N8CCN(C)CC8)ccc7[nH]6)cc5[nH]4)cc3)cc2N)C1]

The phenol compound (2.20 g, 12.1 mmol) is dissolved in acetone (dried through a pad of Na₂SO₄, 48.4 mL) and to this solution is added 1,5-dibromopentane (49.4 mL, 36.3 mmol) and K₂CO₃ (6.69 g, 48.4 mmol). The reaction is heated to reflux under Ar for 6 hrs. The reaction is cooled to RT and the solid is filtered out. The filtrate is concentrated and purified with CombiFlash in 0-30% EtOAc/p-ether to obtained EC1851 (3.3893 g, yield 84.5%) as a solid. LCMS: [M+H]⁺ m/z=331. H NMR (CDCl₃, δ in ppm): 7.65 (dd, J=8.5, 2.0 Hz, 1H), 7.54 (d, J=2.0 Hz, 1H), 6.86 (d, J=8.50 Hz, 1H), 4.08 (t, J=6.50 Hz, 2H), 3.91 (s, 3H), 3.89 (s, 3H), 3.44 (t, J=6.5 Hz, 2H), 1.95 (m, 4H), 1.65 (m, 2H).

EC1851 (3.3893 g, 10.23 mmol) in Ac₂O (52 mL) is cooled to 0° C. and treated with Cu(NO₃).3H₂O (2.967 g, 12.28 mmol) by slow addition. The reaction is stirred at 0° C. for 1 hr then at RT for 2 hrs. After the reaction is completed, the reaction mixture is poured into ice water and stirred for 1 hr. The resultant precipitate is collected by filtration. The product is washed with water (3×) and air-dried as EC1852 (3.7097 g, yield 96%). LCMS: [M+H]⁺ m/z=376. ¹H NMR (CDCl₃, δ in ppm): 7.41 (s, 1H), 7.05 (s, 1H), 4.08 (t, J=6.50 Hz, 2H), 3.94 (s, 3H), 3.89 (s, 3H), 3.42 (t, J=7.0 Hz, 2H), 1.93 (m, 4H), 1.63 (m, 2H).

The solution of EC1852 (37.6 mg, 0.1 mmol) and Hochest dye (53.3 mg, 0.1 mmol) in DMF (1.5 mL) under Ar is treated with K2CO₃ at rt. The reaction is heated to 60° C. and kept for overnight. Then the reaction is cooled to rt and the solid is filtered out. The residue is purified with Prep-HPLC (Mobile phase A: 50 mM NH₄HCO₃ buffer, pH 7.0; B=ACN. Method: 10-100 B % in 30 min.) to afford EC1859 (13.1 mg, yield 18%). LCMS: [M+H]⁺ m/z=720.71.

EC1859 (13.1 mg, 0.0182 mmol) is dissolved in THF/MeOH/H₂O (3/1/1, 0.2 mL) and treated with aq. LiOH solution (1 M, 36 μL) for 4 hrs at rt under Ar. Most of the solvent is removed in vacuo and the aqueous phase is acidified with concentrated HCl to pH 2-3, the precipitate is collected as solid (EC1863, 12.8 mg, without purification) by filtration. The filtrate is washed with water (3×) and air dried for the next step. LCMS: [M+H]⁺ m/z=706.

EC1863 (15.7 mg, 0.022 mmol) in MeOH (10 mL) is subjected to hydrogenation in a Parr shaker (10% wet Pd/C, 5% wt, 7.85 mg, H₂ 41 PSI) for 2 hrs. The product is isolated by filtration through a pad of celite. The solvent is removed in vacuo to give crude EC1870, LCMS: [M+H]⁺ m/z=676.79.

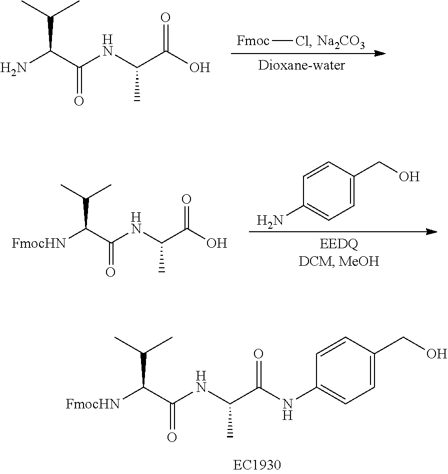

To a solution of Val-Ala-OH (1 g, 5.31 mM) in water (40 ml) is added Na₂CO₃ (1.42 g, 13.28 mM) and cooled to 0° C. before dioxane (40 mL) is added. A solution of Fmoc-Cl (1.44 g, 5.58 mM) in dioxane (40 mL) is added dropwise over 10 min at 0° C. The reaction mixture is stirred at 0° C. for 2 h, then allowed to stir at RT for 16 h. Dioxane is removed under vacuum, the reaction mixture diluted with water (450 mL), pH is adjusted to 2 using 1N HCl and extracted with EtOAc (3×250 mL). The combined organic layers are washed with brine, dried over MgSO₄, filtered, concentrated under reduced pressure and dried to yield Fmoc-Val-Ala-OH. This product is suspended in dry DCM (25 ml), PABA (0.785 g, 6.38 mM) and EEDQ (1.971 g, 7.97 mM) are added. The resulting mixture is treated under Argon with methanol until a clear solution is obtained. The reaction is stirred overnight and filtered. The filtrate is washed with diethyl ether (4×) and dried under high vacum to yield EC1930 (1.85 g, 68%). ¹H NMR (500 MHz, CD₃OD): δ 7.79 (d, J₁=8.0 Hz, 2H), 7.65 (t, J₁=7.0 Hz, J₂=7.5 Hz, 2H), 7.54 (d, J₁=8.0 Hz, 2H), 7.38 (t, J₁=7.5 Hz, J₂=7.5 Hz, 2H), 7.33-7.24 (m, 4H), 4.54 (s, 2H), 4.48 (q, J₁=14.0 Hz, J₂=7.0 Hz, 1H), 4.42-4.32 (m, 2H), 4.22 (t, J₁=7.0 Hz, J₂=6.5 Hz, 1H), 3.94 (d, J₁=7.0 Hz, 1H), 2.07 (m, 1H), 1.43 (d, J₁=7.5 Hz, 3H), 0.97 (d, J₁=7.0 Hz, 3H), 0.95 (d, J₁=7.0 Hz, 3H); LCMS (ESI): (M+H)=Calculated for C₃₀H₃₃N₃O₅, 516.24; found 516.24.

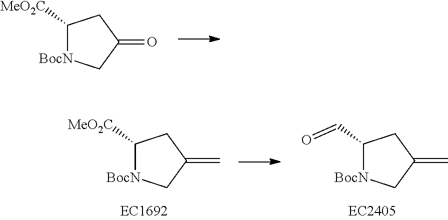 [SMILES: COC(=O)[C@@H]1CC(=O)CN1C(=O)OC(C)(C)C ; C=C1C[C@@H](C(=O)OC)N(C(=O)OC(C)(C)C)C1 ; C=C1C[C@@H](C=O)N(C(=O)OC(C)(C)C)C1]

EC1692. (S)-1-tert-butyl 2-methyl 4-oxopyrrolidine-1,2-dicarboxylate is converted to EC1692 by Wittig reaction. Ph₃PCH₃Br (917.8 mg, 2.57 mmol) in THF (30 mL) is treated with KO^{t}Bu (1 M in THF, 2.57 μL, 2.57 mmol) at 0° C. by dropwise addition. The reaction is kept at ambient temperature for 2 h. Into the stirred solution is added the ketone (250 mg, 1.028 mmol) in THF 20 mL) at 0-10° C. The reaction is then stirred at ambient temperature overnight. The reaction is quenched with H₂O/EtOAc (1:1, 40 mL) and most THF is removed under reduced pressure. The aqueous phase is extracted with EtOAc (20 mL, 3×) and the organic phase is washed with H₂O, and brine sequentially and dried over anhydrous Na₂SO₄ and concentrated. The residue is purified with CombiFlash in 0-50% EtOAc/petroleum ether to give EC1692 (77.2 mg, 31%). LCMS: [M-Boc+H]⁺ m/z=142.

EC2405. (S)-1-tert-butyl 2-methyl 4-methylenepyrrolidine-1,2-dicarboxylate (353.2 mg, 1.46 mmol) in DCM/toluene (1:3, 9.8 mL) is treated with DIBAL (1 M in toluene, 2 eq, 2.92 mmol) dropwise at −78° C. under argon. The reaction is stirred at −78° C. for about 4 h. Then the reaction is quenched with addition of 60 μL of MeOH at −78° C. followed by 5% HCl (0.5 mL) and EtOAc (18 mL). The cold bath is removed and the reaction is stirred for 30 min. The EtOAc layer is separated and washed with brine, dried over anhydrous Na₂SO₄ and concentrated to give EC2405.

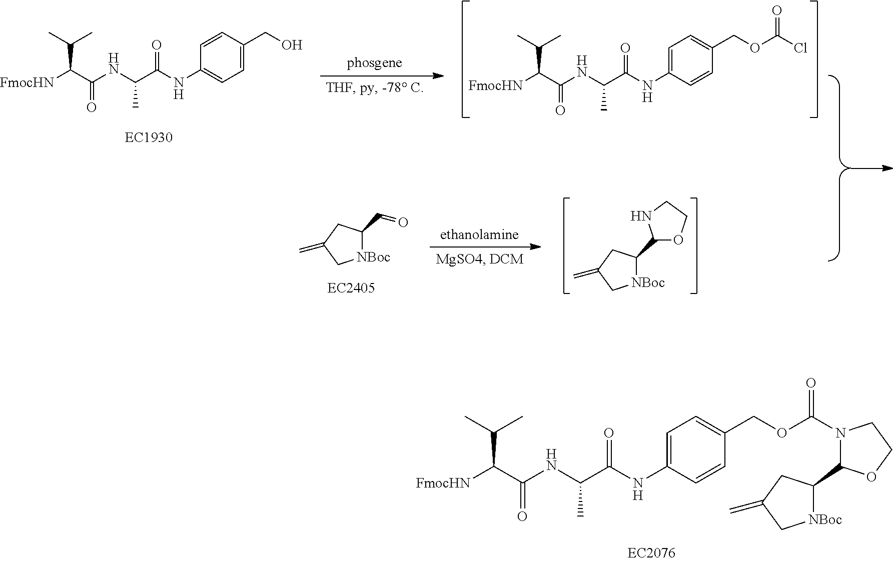

EC2405 (550 mg, 2.6 mmol) is dissolved in DCM (10 mL), and MgSO₄ (3 g) is added followed by dropwise addition of ethanolamine (0.16 mL, 2.6 mmol) in DCM (10 mL). The reaction is stirred at rt for 1 hr. Filtration and concentration under vacuum gave the oxazoline intermediate. In another flask, EC1930 (516 mg, 1.0 mmol) is dissolved in THF (40 mL) and pyridine is added (0.8 mL, 10 mmol). The solution is cooled to −78° C., and diphosgene (0.16 mL, 1.5 mmol) is added. The reaction is stirred at −78° C. for 1 h, DCM (20 mL) and a solution of oxazolidine intermediate is added dropwise. The reaction mixture is allowed to warm to −20° C. over several hours. LC-MS and TLC showed product formation. The reaction mixture is concentrated with silica gel and purified by flash chromatography (120 gold Redisep column, 0-100% EtOAc in petroleum ether) to give EC2076 (0.59 g, 74%). LCMS (ESI): (M+H)+=Calculated for C₄₄H₅₃N₅O₉, 796.38; found 796.74.

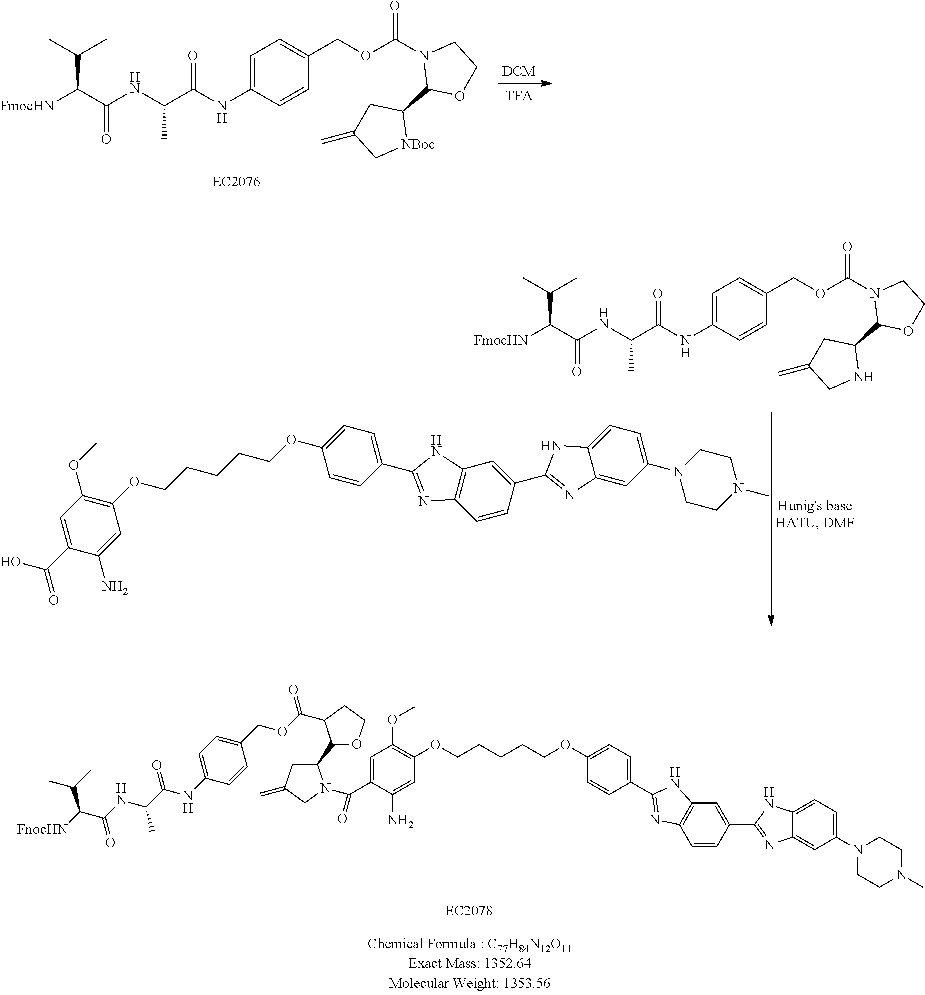

EC2076 (101.0 mg, 0.127 mmol) is stirred in TFA/DCM (0.5 mL each) at rt for 30 min. LC-MS showed complete removal of Boc group. The reaction mixture is concentrated under high vacuum to remove TFA and DCM, re-dissolved in DMF (1.0 mL), and adjusted pH to 8-9 by adding Hunig's base (0.3 mL). EC1870 (86.0 mg, 0.127 mmol) is added, followed by PyBoP (84 mg, 0.16 mmol) and the reaction is stirred at rt for 2 h. LC-MS at 90 min showed that the major peak had the desired product. The reaction mixture is loaded onto a silica gel cartridge and purified by flash chromatography (12 g gold, 0-30% MeOH/DCM) to give desired product, EC2078 (140 mg, 81%). LCMS (ESI): (M+H)⁺=Calculated for C₇₇H₈₄N₁₂O₁₁, 1353.64; found 1354.18.

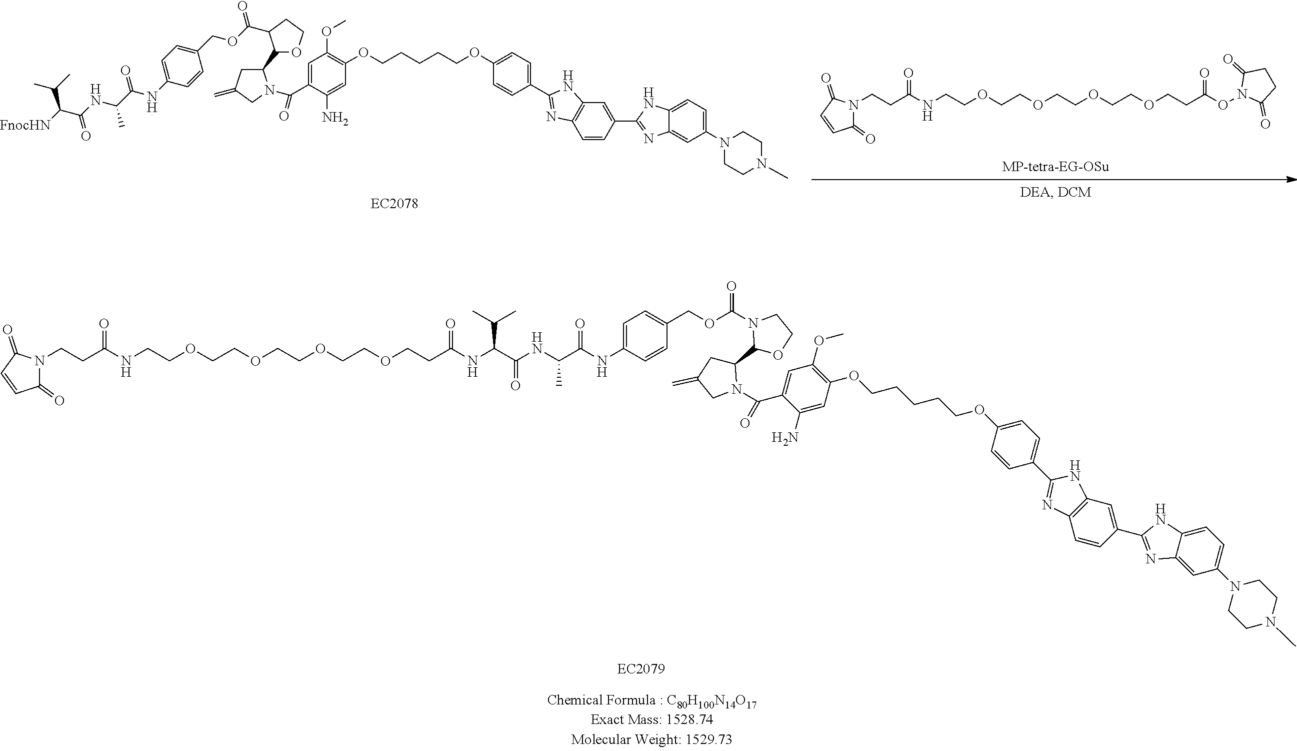

EC2078 (140 mg, 0.10 mmol) is dissolved in DEA/DCM (12/18 mL) and stirred at rt for 30 min. LC-MS showed complete removal of Fmoc group. The reaction mixture is concentrated under high vacuum to remove excess diethylamine and re-dissolved in DCM (5 mL). Commercially available α-Maleimidopropionyl-co-succinimidyl-4(ethylene glycol) (Mal-PEG₄-NHS) (62 mg, 0.12 mmol) is added and the reaction is stirred at rt for 1 hr. The reaction mixture is concentrated, redissolved in DMSO and loaded directly to HPLC column and purified by preparative HPLC (C18 column, 5-80% ACN/pH7 buffer) giving desired product EC2079 (55.8 mg, 36%). LCMS: [M+2H]²⁺ m/z=Calculated for C₈₀H₁₀₀N₁₄O₁₇, 765.37; found 765.74.

EXAMPLE. N¹⁰-TFA Protected EC1579 is prepared according to the following process.

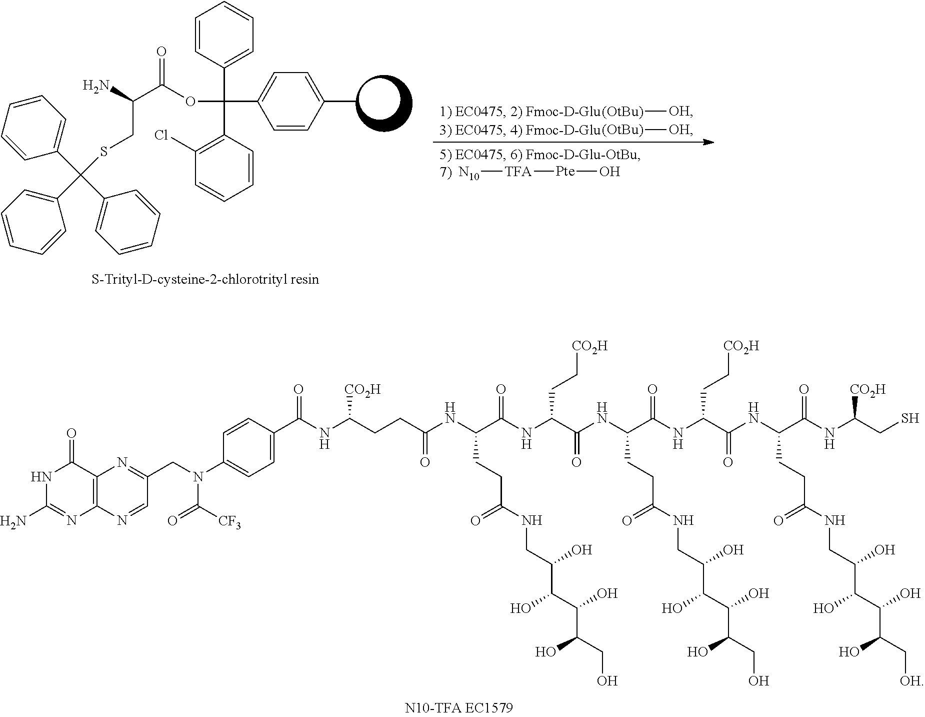

EXAMPLE. EC1579 is described in WO2014/062679. EC1579 is prepared according to the following process.

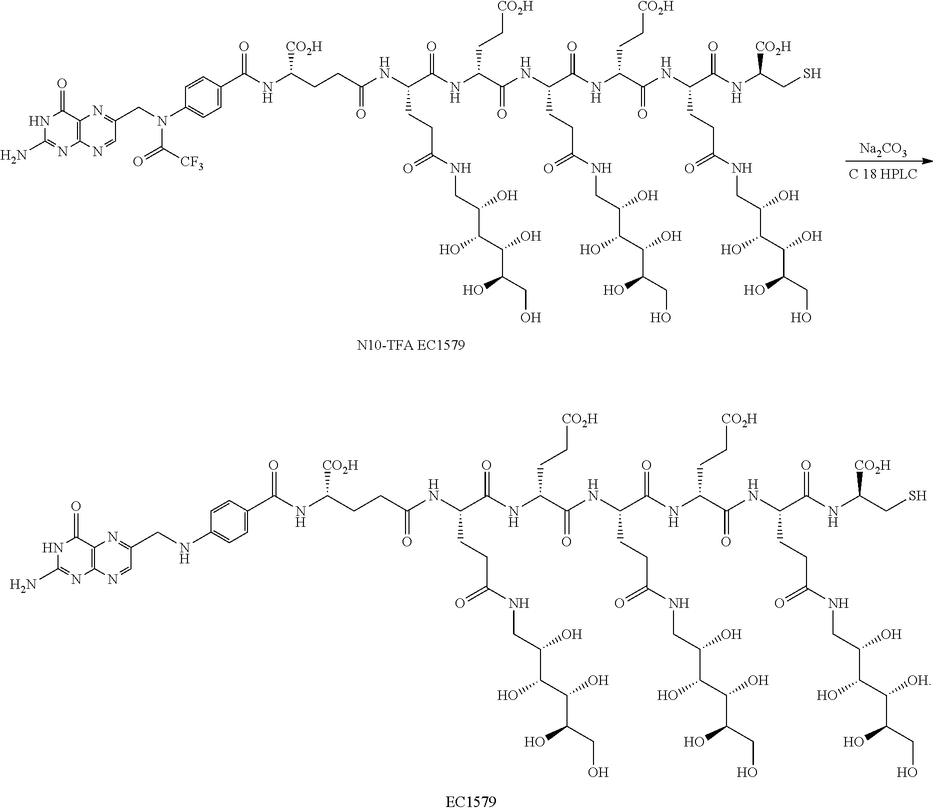

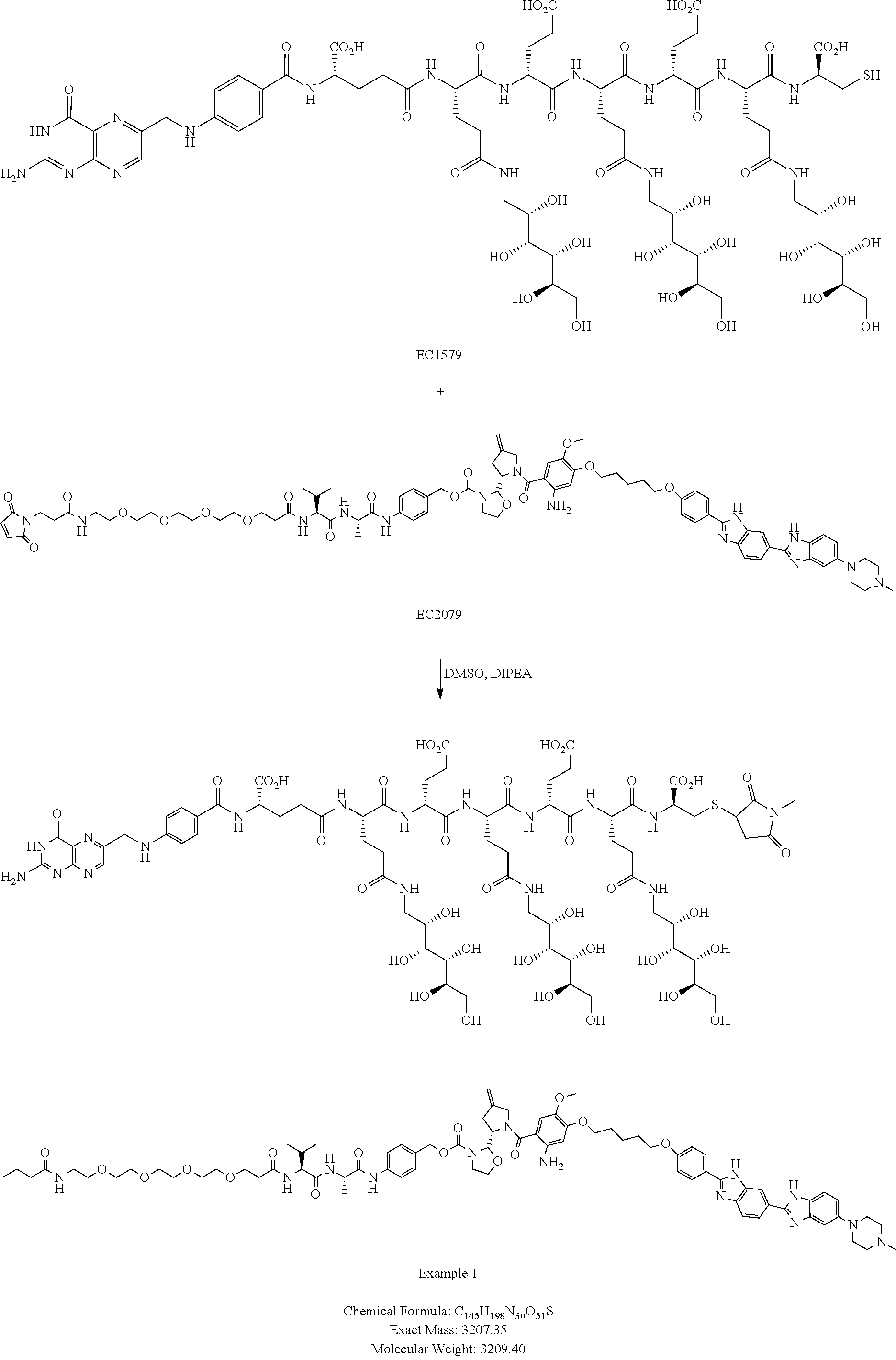

EC1579 (9.85 mg, 0.006 mmol) is stirred in DMSO (2 mL) until dissolved. DIPEA (50 uL) is added, followed by EC2079 (6.24 mg, 0.004 mmol) in DMSO (2 mL). The reaction is stirred at RT for 50 min. LC-MS analysis at 10 min showed complete conversion. The reaction mixture is directly loaded on a prep-HPLC column and purified (10-100% MeCN/Ammonium bicarbonate, pH 7 buffer) to give desired product Example 1 (5.5 mg, 42%). ¹H NMR (500 MHz, DMSO-D₆+D₂O) (selected data): δ 8.60 (s, 1H), 8.44-8.08 (m*, 1H), 8.07 (d, J=8.5 Hz, 2H), 8.06-7.84 (m*, 2H), 7.80-7.57 (m*, 2H), 7.57 (d, J=8 Hz, 2H), 7.51 (d, J=6.5 Hz, 2H), 7.44 (m*, 1H), 7.22 (m*, 2H), 7.08 (d, J=8 Hz, 2H), 6.93 (d, J=8.5 Hz, 1H), 6.60 (d, J=8.5 Hz, 2H), 6.33 (s, 1H), 4.95 (m*, 4H), 4.45 (m*, 3H); LCMS: [M+4H]⁴⁺ m/z=Calculated for C₁₄₅H₁₉₈N₃₀O₅₁S, 803.34; found 803.80.

1. A method for treating a cancer comprising the steps of identifying the presence of tumor-associated macrophages in the cancer in a host animal, and administering to the host animal a therapeutically effective amount of one or more compounds comprising a folate receptor binding compound attached to a drug via a linker.

2. A method for treating a cancer in a host animal, the method comprising the step of administering to the host animal a therapeutically effective amount of one or more compounds comprising a folate receptor binding compound attached to a drug via a linker to inhibit or deplete tumor-associated macrophages in the host animal.

3. A method for targeting tumor-associated macrophages in a host animal, the method comprising the step of administering to the host animal a therapeutically or diagnostically effective amount of one or more compounds comprising a folate receptor binding compound attached to a drug via a linker to target the tumor-associated macrophages.

4. The method of claim 1, wherein the folate receptor binding compound is specific for the folate receptor-β.

5.-8. (canceled)

9. The method of claim 1, wherein the tumor-associated macrophages are in the cancer and the tumor-associated macrophages are pro-tumor M2-biased and express one or more markers selected from the group consisting of CD163(+), IL10(+), Arg1(+), TGF-β(+), VEGF(+), and CD206(+).

10. (canceled)

11. The method of claim 1, wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis.

12. (canceled)

13. The method of claim 1, wherein the drug is selected from the group consisting of trabectedin, doxorubicin, gemcitabine, a bisphosphonate, and a proapoptotic peptide.

14.-16. (canceled)

17. The method of claim 1, wherein the drug is selected from the group consisting of a TLR9 agonist, a TLR3 agonist, a TLR7/8 agonist, a monophosphoryl lipid A, a mTOR inhibitor, a PPARγ agonist, and a PPARδ agonist.

18.-25. (canceled)

26. The method of claim 1, wherein the drug is selected from the group consisting of silibinin, a src kinase inhibitor, a MerTK inhibitor, and a Stat3 inhibitor.

27.-41. (canceled)

42. The method of claim 2, wherein the folate receptor binding compound is specific for folate receptor-β.

43. The method of claim 3, wherein the folate receptor binding compound is specific for folate receptor-β.

44. The method of claim 2, wherein the tumor-associated macrophages are in the cancer and the tumor-associated macrophages are pro-tumor M2-biased and express one or more markers selected from the group consisting of CD163(+), IL10(+), Arg1(+), TGF-β(+), VEGF(+), and CD206(+).

45. The method of claim 3, wherein the tumor-associated macrophages are in the cancer and the tumor-associated macrophages are pro-tumor M2-biased and express one or more markers selected from the group consisting of CD163(+), IL10(+), Arg1(+), TGF-β(+), VEGF(+), and CD206(+).

46. The method of claim 2, wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis.

47. The method of claim 3, wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis.

48. The method of claim 2, wherein the drug is selected from the group consisting of a DNA-alkylating agent, a pyrrolobenzodiazepine, trabectedin, doxorubicin, gemcitabine, a bisphosphonate, and a proapoptotic peptide.

49. The method of claim 3, wherein the drug is selected from the group consisting of a DNA-alkylating agent, a pyrrolobenzodiazepine, trabectedin, doxorubicin, gemcitabine, a bisphosphonate, and a proapoptotic peptide.

50. The method of claim 2, wherein the drug is selected from the group consisting of a TLR9 agonist, a TLR3 agonist, a TLR7/8 agonist, a monophosphoryl lipid A, a mTOR inhibitor, a PPARγ agonist, and a PPARS agonist.

51. The method of claim 3, wherein the drug is selected from the group consisting of a TLR9 agonist, a TLR3 agonist, a TLR7/8 agonist, a monophosphoryl lipid A, a mTOR inhibitor, a PPARγ agonist, and a PPARS agonist.

52. The method of claim 2, wherein the drug is selected from the group consisting of silibinin, a src kinase inhibitor, a MerTK inhibitor, and a Stat3 inhibitor.

53. The method of claim 3, wherein the drug is selected from the group consisting of silibinin, a src kinase inhibitor, a MerTK inhibitor, and a Stat3 inhibitor.


SEQUENCE LISTING (1 sequence)

SEQ ID NO: 1 - protein, 14 aa, Artificial Sequence
  Synthetic polypeptide
KLAKLAKKLAKLAK